(19) United States
(12) Patent Application Publication
(10) Pub. No.: US 2011/0011917 A1
(43) Pub. Date: Jan. 20, 2011

(54) METHODS, DEVICES, AND KITS FOR TREATING VALVE PROLAPSE

(75) Inventor: Didier Loulmet, New York, NY (US)

Correspondence Address: VISTA IP LAW GROUP LLP, 12930 Saratoga Avenue, Suite D-2, Saratoga, CA, 95070, US

(73) Assignee: Hansen Medical, Inc., Mountain View, CA (US)

(21) Appl. No.: 12/651,287

(22) Filed: Dec. 31, 2009

Related U.S. Application Data
(60) Provisional application No. 61/142,076, filed on Dec. 31, 2008.

(51) Int. Cl. A61B 17/064

(52) U.S. Cl. 227/181.1

(57) ABSTRACT

Methods, devices, and kits are described for addressing cardiac valve repair. A system for deploying a tensile member between two tissue structures may comprise a first anchor insertion instrument defining a working lumen therethrough, and a second anchor insertion instrument movably coupled through the working lumen, wherein the first anchor instrument defines a jaw space and is configured to deploy a staple prosthesis into a portion of a first tissue structure which may be captured within the jaw space, and wherein the second anchor instrument is configured to deploy an implantable anchor, such as an anchor dart, into a second tissue structure which is coupled to the staple prosthesis by a tensile member, such as a suture.

### RELATED APPLICATION DATA

The present application claims the benefit under 35 U.S.C. §119 to U.S. provisional patent application Ser. No. 61/142,076, filed Dec. 31, 2008. The foregoing application is hereby incorporated by reference into the present application in its entirety.

### FIELD

The methods, devices, and kits described here are in the field of cardiac valve repair, and more specifically, in the field of treating valve prolapse.

### BACKGROUND

The mitral valve comprises two leaflets attached to the mitral valve annulus, which are supported towards their free edge by cords (chordae tendinae) fastened to the internal wall of the ventricle and to the papillary muscles. However, sometimes one or both of the valve leaflets become loose, due to failure or loosening of one or several of these cords. The valve then prolapses and its bloodtight seal becomes compromised, causing the blood to flow back into the left atrium during systole.

Some solutions to this prolapse problem have focused on either replacing the whole valve with an artificial one, or repairing the part of the valve that is diseased in order to restore normal function. Other solutions focus on clipping the valve leaflets together in order to obtain better leaflet coaption. Most of these solutions are surgical, as opposed to percutaneous, in nature, requiring an incision into the thoracic cavity (e.g., a median sternotomy) and into the heart. This type of surgery also necessitates arresting the heart, and thus the use of a extracorporeal circulation system such as a heart-lung-bypass machine to take over the heart function while the patient's heart is arrested. This surgery is incredibly invasive, causing high risks and morbidity to those eligible. In addition, the use of a heart-lung-bypass machine poses an inflammatory reaction risk as components of the blood can get activated while circulating in the machines tubes, reservoirs, pumps, and oxygenators, which are made of foreign materials. Because of the risks and invasiveness of the surgery, the recovery time is typically quite lengthy.

Accordingly, it would be advantageous to have methods, devices, and kits for treating mitral valve prolapse, which are less invasive and pose less risks to the patient than typical open heart surgery. It would also be advantageous to have alternative methods and devices for treating mitral valve prolapse.

### SUMMARY

One embodiment is directed to a system for deploying a tensile member between two tissue structures, comprising a first anchor insertion instrument defining a working lumen therethrough; a second anchor insertion instrument movably coupled through said working lumen; wherein the first anchor instrument defines a jaw space and is configured to deploy a staple prosthesis into a portion of a first tissue structure which may be captured within said jaw space; and wherein the second anchor instrument is configured to deploy an implantable anchor into a second tissue structure which is coupled to the staple prosthesis by a tensile member. The jaw space of the first anchor insertion instrument may be defined distally by an anvil structure. The jaw space of the first anchor insertion instrument may be defined proximally by a structure selected from the group consisting of a proximal stapler housing, an advanceable stapling member, and an advanceable jaw member. A portion of the first tissue structure may be controllably compressed by the advanceable jaw member before a staple is advanced through such first tissue structure portion by the advanceable stapling member, thereby driving the staple through the portion of the first tissue structure. The implantable anchor may comprise an anchor dart having a distal body portion and a plurality of legs defining a proximal portion. A lumen may be defined through said body portion. A working space may be defined in between the plurality of legs. The second anchor insertion instrument may comprise an elongate probe having a distal end removably inserted through the working space and body portion lumen of the anchor dart. The second anchor insertion instrument may comprise an elongate probe defining a working lumen through which the anchor dart may be advanced into the second tissue structure. The distal portion of the elongate probe may comprise a sharp geometry configured for atraumatic advancement through the second tissue structure before deployment of the anchor dart.

Another embodiment is directed to a method for deploying a tensile member between two tissue structures, comprising navigating a unitary delivery instrument assembly defining a working lumen to a position adjacent a first targeted tissue structure; deploying an implantable anchor dart prosthesis into the first targeted tissue structure, the anchor dart prosthesis being coupled to a tensile member which remains coupled to the delivery instrument assembly; navigating the unitary delivery instrument assembly to a position adjacent a second targeted tissue structure; deploying a second anchor into the second targeted tissue structure which remains coupled to the anchor dart prosthesis by the tensile member; adjusting the length of the tensile member between the anchor dart and second anchor; and removing the unitary delivery instrument assembly. The first targeted tissue structure may be a portion of a papillary muscle. The second targeted tissue structure may be a portion of a mitral valve leaflet. Navigating a unitary delivery instrument may comprise operating a remotely steerable catheter. Deploying an implantable anchor dart prosthesis may comprise inserting a deployment probe having a sharp distal tip and a working lumen in which the implantable anchor dart prosthesis is disposed. Deploying an implantable anchor dart prosthesis may further comprise advancing the anchor dart prosthesis distally relative to the deployment probe. Deploying an implantable anchor dart prosthesis may comprise inserting a deployment probe having a sharp distal tip positioned through a proximal workspace and body lumen defined by the anchor dart prosthesis, such that the sharp distal tip extends beyond a distal tip of the anchor dart prosthesis. Deploying an implantable anchor dart prosthesis may further comprise inserting the deployment probe sharp distal tip into the first targeted tissue structure with a relatively high impulse load/timing profile. Deploying an implantable anchor dart prosthesis may further comprise withdrawing the deployment probe proximally relative to the implantable anchor dart. Deploying a second anchor may comprise deploying a staple.

### BRIEF DESCRIPTION OF THE DRAWINGS

FIG. 1 depicts one method of accessing a prolapsed leaflet of a mitral valve using a trans-septal catheter, in accordance with the methods and devices herein described.

FIG. 2 provides a magnified view of FIG. 1, where a first catheter has been introduced through the trans-septal catheter and into the left atrium.

FIG. 3 depicts the application of suction from the first catheter to the papillary muscle.

FIG. 4A provides a magnified view of the distal end of the first catheter, which is temporarily adhered to the papillary muscle via suction.

FIG. 4B provides an illustrative example of a suitable first anchor, in accordance with the methods, devices, and kits described herein.

FIGS. 4C-D provide views of an illustrative example of another suitable anchor embodiment.

FIG. 4E illustrates two deployment configurations for utilizing an anchor such as that illustrated in FIGS. 4C-D.

FIG. 4F illustrates a delivery instrumentation configuration which may be utilized to deploy an anchor such as that illustrated in FIGS. 4C-D.

FIG. 5 provides a magnified view of the deployment of the first anchor into the papillary muscle tissue.

FIG. 6 depicts the cord secured to the papillary muscle via the first anchor, as the first catheter is being removed.

FIG. 7 provides an illustration of the second anchor being advanced to the proximity of the prolapsed mitral valve leaflet.

FIG. 8A provides an enlarged view of a suitable second anchor as described herein.

FIGS. 8B-8F illustrate various views of a compact stapling assembly.

FIGS. 8G-8K illustrate various views of an assembly configured to deliver a first anchor to one tissue structure, as well as a second anchor to another tissue structure, with a tensile member in between, such as a suture.

FIG. 9 shows an illustrative method of positioning the second anchor adjacent to the prolapsed mitral valve leaflet.

FIG. 10 shows an illustrative method of deploying the second anchor into the prolapsed mitral valve leaflet.

FIG. 11 provides an illustrative depiction of the advancement of a third catheter carrying a fastener.

FIG. 12 shows the fastener being positioned and deployed adjacent to the second anchor, after the prolapsed valve has been positioned and the cord has been pulled to tension the cord so that the prolapse has been corrected.

FIG. 13 shows the release of the fastener and removal of the third catheter.

FIG. 14 provides an illustrative depiction of the advancement of a cutting wire.

FIG. 15 shows the removal of the cutting wire after excess cord has been cut immediately proximal to the fastener.

FIGS. 16A-16C illustrate a cutting wire cutting a cord.

FIGS. 17A-27C illustrate various embodiments of the present invention for deploying a prosthetic chordae tendinae cord.

### DETAILED DESCRIPTION

Described here are methods, devices, and kits for treating valve prolapse (such as mitral valve prolapse). In general, the methods, devices, and kits relate to the use of a cord that can be secured into cardiac tissue located below the valve leaflets (e.g., the papillary muscles or the ventricular wall), coupled to a prolapsed valve leaflet, and then tensioned to correct the prolapse. While the majority of the description herein relates to the use of these devices and kits during a percutaneous or intravascular procedure, the devices and kits may also be used during open surgical procedures as well.

### Devices

In one combination, the devices for treating a prolapsed mitral valve leaflet comprise a flexible cord, having a proximal end and a distal end, a first anchor attached to the cord at its distal end, and a second anchor slidably attached to the cord. In some variations, the first anchor is configured to secure the cord to cardiac tissue located below the prolapsed mitral valve leaflet, and the second anchor is configured to secure into the prolapsed mitral valve leaflet. In some variations, the first anchor is configured to secure into the prolapsed mitral valve leaflet, and the second anchor is configured to secure the cord to cardiac tissue located below the prolapsed mitral valve leaflet.

As will be described in more detail below, the first anchor may have a tissue piercing tip, which in some variations comprises two legs that may be configured to expand after securing into cardiac tissue (e.g., a papillary muscle and a ventricular wall). Similarly, the second anchor may comprise two legs that are configured to pierce mitral valve leaflet tissue, and may further comprise an eyelet. Either of the anchors may be made from a shape memory material such as a nickel titanium alloy.

In some embodiments, one of more anchor structures may comprise bioresorbable or biodegradable materials, such as polymers, including but not limited to polylactic acid (PLA), polygolic acid (PGA), polycaprolactone (PCL), polydoxanone (PDO), poly trimethylene carbonate (TMC), poly-l-lactic acid (PLLA), and copolymers of these. In operation, anchor structures comprising such materials may be implanted with a non-resorbable cord, and then over time such materials resorb away, leaving a non-resorbable cord prosthesis preferably anchored to tissue rather than the anchor structures, in a configuration closely mimicking that of the pre-intervention tissue structures.

The cord is typically flexible so that it can be maneuvered through a catheter, and may, for example, be made from a material selected from the group consisting of non-polymeric fabrics, polymers, or mixtures thereof. In some variations, the cord is made from a polymeric and non-polymeric fabric mixture, such as a PTFE (polytetrafluoroethylene, including expanded polytetrafluoroethylene) fabric, although any suitable artificial or biological material may be used. It should be noted that the cord is of such a length as to enable it to be manipulated outside of the patient's body. Thus, the suitable length of the cord is determined in large part by the method of access (e.g., less cord will be necessary if the prolapsed mitral valve leaflet is accessed via the jugular rather than the femoral).

In some embodiments, it is desirable to visualize a prosthetic cord utilizing imaging modalities such as ultrasound and fluoroscopy. In embodiments wherein fluoroscopic visualization is desired, the cord may be fitted with one or more radioopaque markers in the form of small beads of radioopaque metals (platinum, for example) or other materials fastened to one or more locations along the cord. In embodiments wherein ultrasound visualization is desired, portions, or the entirety, of the cord may be coated with echogenic coatings configured to optimize ultrasound visualization. In one embodiment, the coating may be configured to wash away from the prosthetic cord after a limited exposure to blood in situ, such as one day post-operation. In one embodiment, it is desirable to optimize both ultrasound and fluoroscopic visualization, and thus radioopaque marking and echogenic coating may be employed upon a given cord prosthesis.

It is illustrative to note that in the drawings accompanying this description, instruments, such as catheters and push or pull wires, and prosthetic materials, such as prosthetic cords, are manipulated during the illustrative procedures from outside of the body (extracorporeally). In other words, while the drawings depict aspects of the heart and other structures, it is illustrative to reiterate that the proximal portions of various of the interventional structures lead from the distal location in or adjacent to the chambers of the heart, through various passageways, such as through blood vessels, then transcutaneously to an extracorporeal location where they may be manipulated by a surgeon or operator. For example, in various illustrative embodiments described herein, until the final fastening and clipping of cord prostheses (elements numbered 400 and 3008, for example), the proximal ends of such prostheses lead proximally to an extracorporeal location where they may be manipulated by the operator or surgeon (for example, the surgeon may tension a cord proximally, thereby providing a tension all the way to the distal anchoring position, which, for example, may be a papillary muscle, leaflet, or other structure; this is a useful variable for the surgeon in adjusting the length of a tension member in-situ, such as a chordae tendinae prosthetic cord, to provide desirable heart valve mechanics), or utilized by such operator or surgeon to pass or guide other instruments from such proximal location to the internal (endocorporeal) interventional site.

The device may further comprise a fastener. In this way, after the cord has been secured into cardiac tissue by the first anchor, and coupled to a prolapsed mitral valve leaflet by a second anchor, it may be pulled tight or tensioned, and fastened in place. For example, the cord may be fastened at a tension that helps correct the valve leaflet prolapse after the valve has been repositioned, as described herein. A more detailed description of the device and all of its optional components will be provided in the discussion of the methods below.

### Methods

In general, the methods for treating a prolapsed valve leaflet comprise securing a first anchor to cardiac tissue located below the prolapsed valve leaflet (wherein the first anchor is attached to a flexible cord), securing a second anchor into the prolapsed valve leaflet (wherein the second anchor is attached to the flexible cord), and securing the cord.

When a catheter is used to apply the device to treat a prolapsed valve, the application may be performed either anterograde (e.g., in the usual direction of flow of blood, typically above the valve) or retrograde (e.g., in the direction against the normal flow of blood, typically from blow the valve). Although most of the examples of devices and methods described herein illustrate the anterograde approach, it should be understood that the retrograde approach may also be used.

One variation of the method for treating a prolapsed valve leaflet (e.g., a mitral valve leaflet) comprises advancing a flexible cord having a first anchor attached at its distal end to the proximity of the prolapsed valve leaflet, securing the first anchor to cardiac tissue located below the prolapsed valve leaflet (e.g., papillary muscle or the ventricular wall), securing a second anchor slidably attached to the cord into the prolapsed mitral valve leaflet, and tensioning the cord after the second anchor has been secured into the prolapsed mitral valve leaflet. The prolapse of the valve leaflet may be corrected so that it does not prolapse by moving the valve into the correct non-prolapsed position (e.g., by pushing against the anchor with a fastener, and by tensioning the cord between the anchors so that the valve leaflet does not prolapse). The cord may be secured in using a fastener. The valve leaflet may be moved by manipulating the cord, by using a catheter, a probe, or by any other appropriate positioning step. For example, the distal end of a catheter may be used to position the valve leaflet, or the fastener may be used to position the valve leaflet. In some variations the valve leaflet is manipulated after an anchor has been inserted into (or through) the valve leaflet, but before the tension on the cord is secured by the fastener. Once the leaflet is positioned, the cord may be optimally tensioned to maintain the corrected position, and the cord may be secured into place and cut.

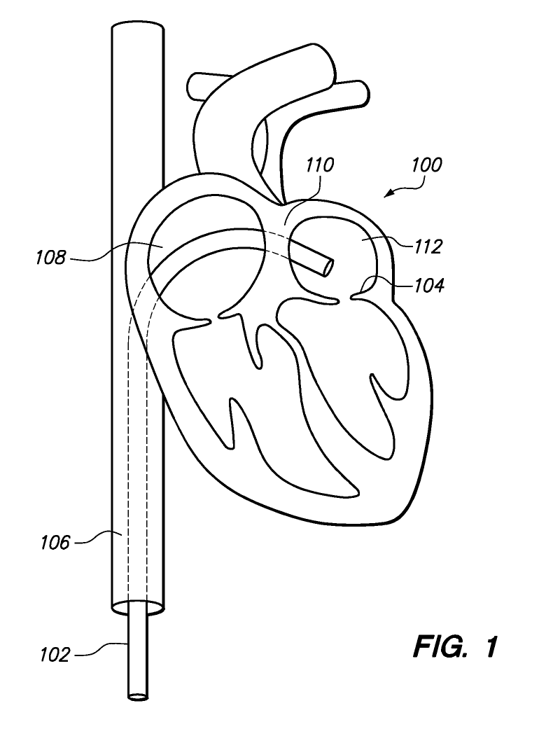

For example, FIG. 1. shows a cross-section of heart (100), having a prolapsed mitral valve leaflet (104). To access the prolapsed mitral valve leaflet (104), a trans-septal catheter (102) is introduced into the inferior vena cava (106), which is in turn accessed through one of the femoral veins. The trans-septal catheter (102) is then advanced up through the right atrium (108) and through the interatrial septum (110). Examples of the trans-septal approach are described in U.S. Pat. No. 6,743,239, U.S. Pat. No. 6,695,866 (herein incorporated by reference in their entirety). The catheter should be flexible and steerable, so that it can be maneuvered through the tortuous anatomy of the vasculature. For example, the catheter may include a steerable sheath, wherein at least part of the sheath (e.g., the distal end) is steerable in one or more directions. Thus, the sheath may be inserted trans-septally and oriented (e.g., towards the anterior commissure) so that another catheter may pass through the sheath and be further steered towards the valve leaflet and/or the cardiac tissue located beneath prolapsed valve leaflet.

As described above, FIGS. 1-15 depict one method of accessing the prolapsed mitral valve leaflet (104) (e.g., the anterograde approach), however different methods of access are also suitable. For example, a catheter may also be introduced via the jugular vein, may be introduced through the right femoral artery, and advanced up to the left atrium (112) by crossing the aortic valve, or may be introduced via the carotid or subclavian arteries. Thus, the order of the steps described in the method may be adapted to suit these variations. For example, in a retrograde approach the valve is accessed from below, and the distal end of the cord is attached to a first anchor adapted to secure to the valve leaflet, and the second anchor is adapted to secure to the cardiac tissue located beneath prolapsed valve leaflet and be slideably connected to the cord.

Any appropriate visualization technique may be used to help the practitioner visualize the valve anatomy, and to manipulate or steer the catheters. For example, intracardiac echo, or transesophageal echo may be used. Thus, the devices described herein may be adapted to enhance visualization of the devices when used with any of the techniques. For example, the devices may include contrasting agents, and they may include electron dense or radioopaque regions, etc. In addition, rapid ventricular pacing, or adenosine IV administration may allow for transient and reversible cardiac arrest in order to stabilize the leaflets and papillary muscles and facilitate targeting.

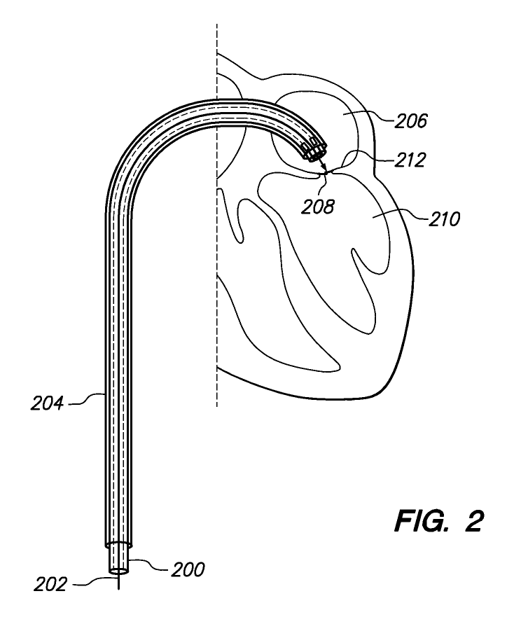

FIG. 2 provides a magnified view of the heart cross-section of FIG. 1. Shown in FIG. 2 is a catheter (200) introduced into trans-septal catheter (204) (e.g., a trans-septal sheath). Catheter (200) is carrying a flexible cord (202). Catheter (202) is advanced through the trans-septal catheter (204) to the left atrium (206), and down through the mitral valve opening (208), and into the left ventricle (210). In some variations, the trans-septal sheath 204 has a steerable (or directional) tip to help guide the catheter(s) towards the leaflet and cardiac tissue beneath the leaflet. As can be seen in FIG. 2, mitral valve leaflet (212) is prolapsed so that it can no longer prevent the back flow of blood into the left atrium when the ventricle contracts.

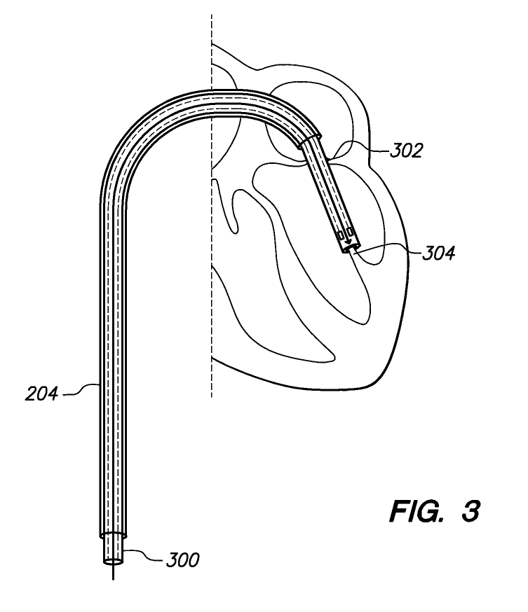

FIG. 3 shows catheter (300) being advanced to cardiac tissue located beneath prolapsed mitral valve leaflet (302), and specifically to the head of papillary muscle (304). In some variations, the catheter may be steerable (e.g., the tip may be at steerable in at least one direction). Thus, the trans-septal sheath (204) and the catheter together may be used to guide the catheter (300). The catheter (300) is connected to a vacuum source (not shown) located outside of the patient's body. In some variations, the edges of the distal end of catheter (300) are designed so as to maximize contact with the papillary muscle and to minimize aspiration of blood into the catheter during the time suction is applied. For example, the distal end of the catheter may be adapted to conform to the cardiac tissue that it contacts (e.g., the head of the papillary muscle). It should be noted, however, that while FIG. 3 depicts catheter (300) being advanced to the head of papillary muscle 304), that the catheter may be advanced to any suitable cardiac tissue located below the prolapsed mitral valve (302). For example, catheter (300) may be advanced to a location along the side or base of the ventricular wall. That is, the catheter need not be advanced to a location directly vertical of the prolapsed mitral valve leaflet.

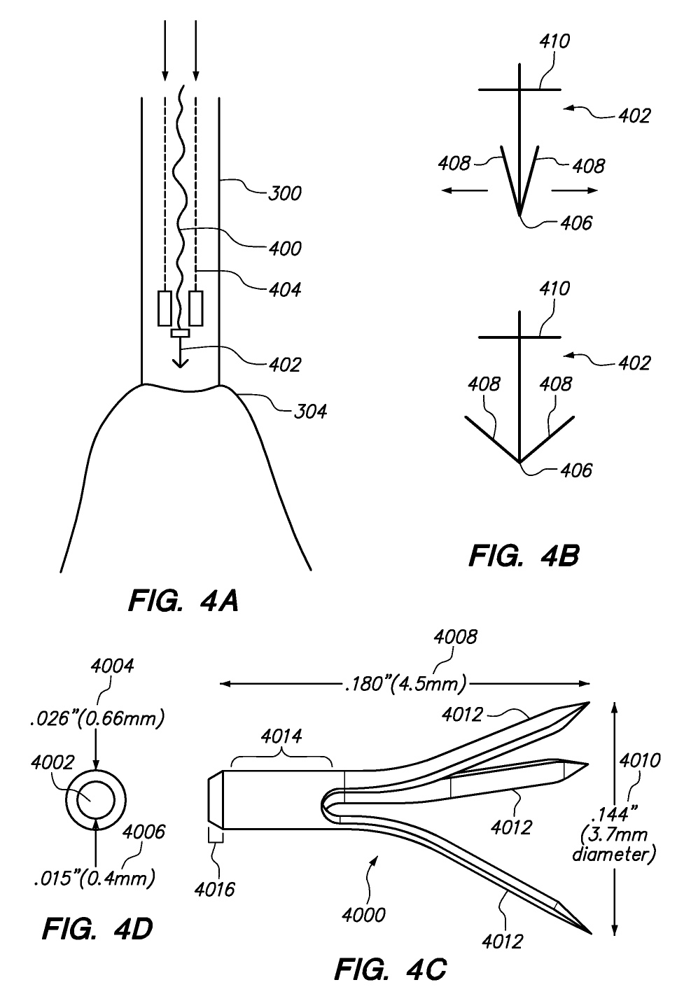

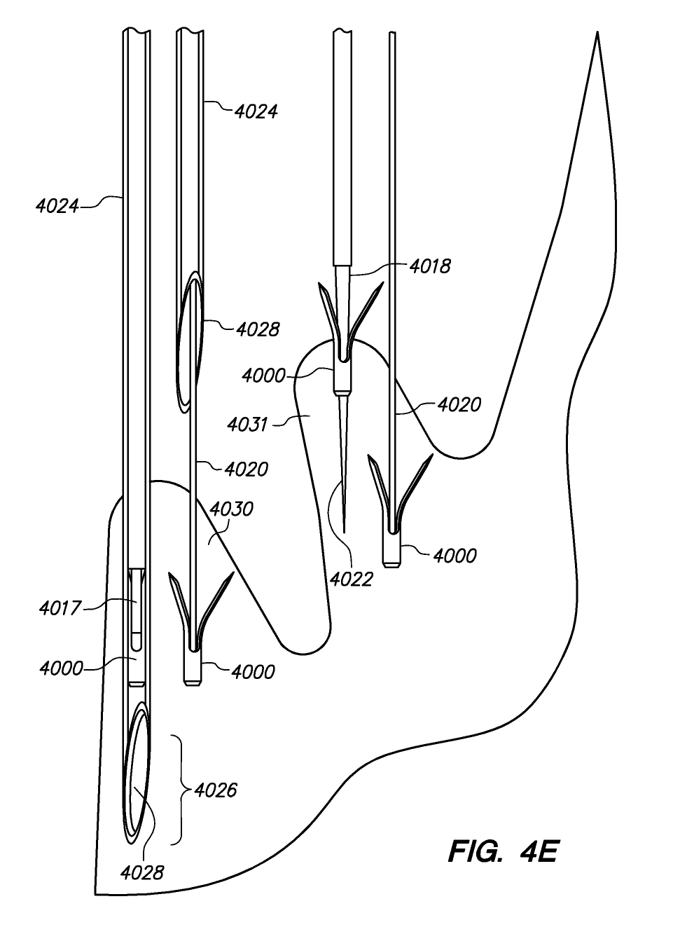

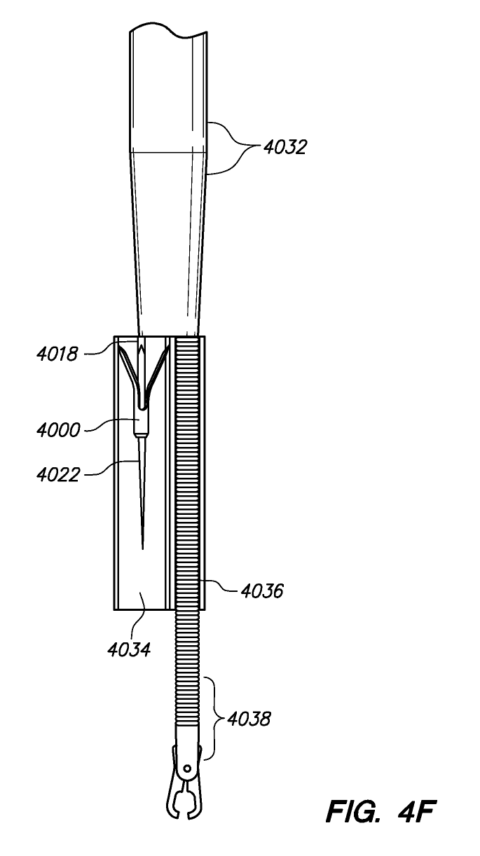

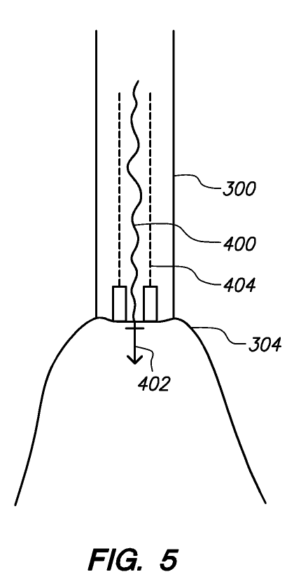

FIG. 4A provides a magnified view of the distal end of the catheter (300) from FIG. 3. As shown there, catheter (300) has been advanced to the head of papillary muscle (304), and is held in place by light suction. In general, adequate suction will be applied to form a seal between the catheter and the head of the papillary muscle. The suction is sufficient to stabilize the catheter to the head of the papillary muscle, but does not damage or otherwise harm the papillary muscle, as would be observed by an ordinary artisan. Catheter (300) is carrying cord (400), which has a papillary, or “first” for the purposes of the embodiments illustrated in FIGS. 1-16C (and thereafter a “papillary anchor (402)”), anchor (402) at its distal end. The first anchor (402) may be secured to the cord (400) in any suitable manner. For example, the cord (400) may be tied to first anchor (402), or first anchor (402) may be welded, bonded, or otherwise adhered to cord (400). Also shown is a plunger or piston-like mechanism (404), which when advanced distally (shown by arrows), deploys first anchor (402) out from the distal end of catheter (300) and into the head of papillary muscle (304). The plunger or piston-like mechanism is typically maneuvered by the practitioner, outside of the patient's body. While a plunger, or piston-like mechanism is shown in FIG. 4A, any suitable deployment mechanism may be used to deploy first anchor (402). For example, a hydraulic mechanism may be used, or the anchor may be deployed with the use of an expandable balloon.

FIG. 4B provides a magnified view of the first anchor (402) shown in an unexpanded (top view) and expanded (bottom view) configuration. As shown by FIG. 4B, first anchor (402) has a tissue piercing tip (406). While the tissue piercing tip (406) of first anchor (402) is shown as being sharp or pointed, it need not be. Indeed, any tip configured to pierce tissue (whether blunt or sharp) is suitable. The tip of first anchor (402) in the variations shown in FIGS. 4A and 4B comprise two legs (408), which are configured to expand after they are secured into cardiac tissue (as shown by the arrows in the top picture in FIG. 4B, and as shown expanded in the bottom picture in FIG. 4B). The legs of the anchors described herein may be extended passively, actively or both to help secure the anchor in the tissue. For example, the legs may be extended actively by releasing them from tension (e.g., compression) or by using a shape-memory alloy, as described above. The legs may be passively extended after insertion into the tissue by pulling “backwards” on the anchor after it has been inserted into the tissue; the legs of the anchor may act as barbs, and expand into the tissue.

First anchor (402) may comprise any suitable number of legs. Indeed, in some variations, it might be preferable for first anchor (402) to have one, two, three or more legs. First anchor (402) is also shown as having a t-bar (410) near its proximal end, although it need not have one. Having a t-bar (410) at its proximal end may help further secure the anchor into cardiac tissue. In some variations the t-bar may provide a surface against which force can be applied to drive the anchor into a tissue. It may also be beneficial to have at least a portion of the first anchor (402) made from a shape memory material such as a nickel titanium alloy. However, any other flexible, yet sturdy, biocompatible material may be used. While first anchor (402) is shown in FIGS. 4A and 4B as having a modified shipping anchor configuration (i.e., having a v-shaped distal end with a t-bar), any suitable configuration may be used. Typically, first anchor (402) is of a size suitable to secure into cardiac tissue. For example, the anchor may be between about 3 and about 12 mm long, or between about 5 and about 10 mm long. In some variations, the anchor may be about 8 mm long. In some variations, the first anchor is longer than it is wide.

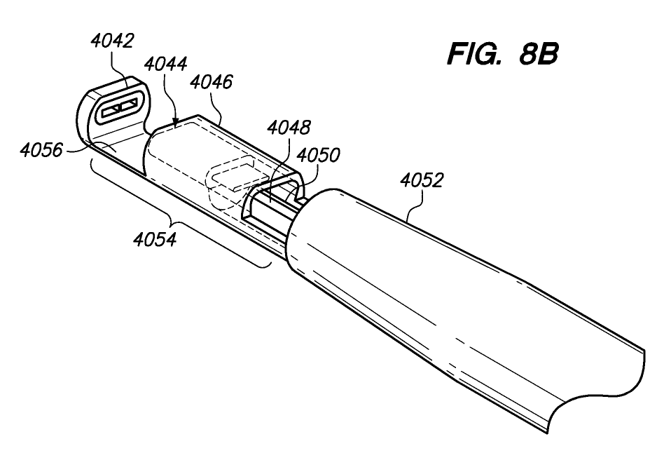

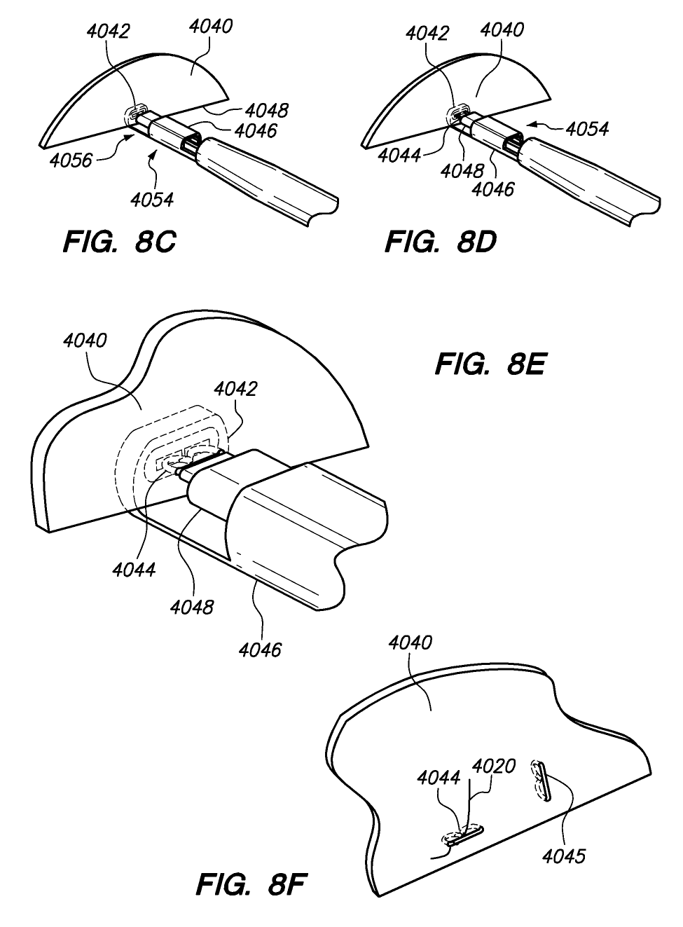

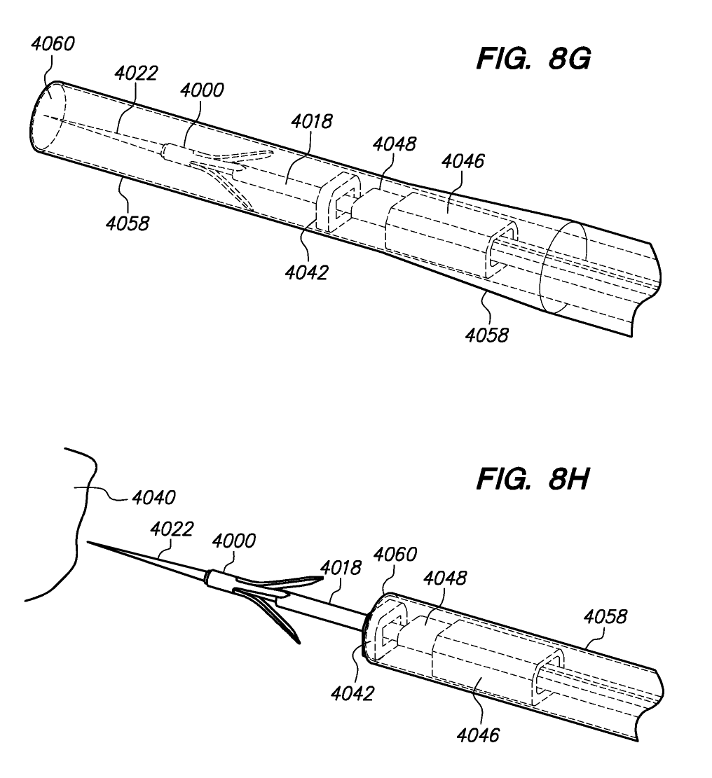

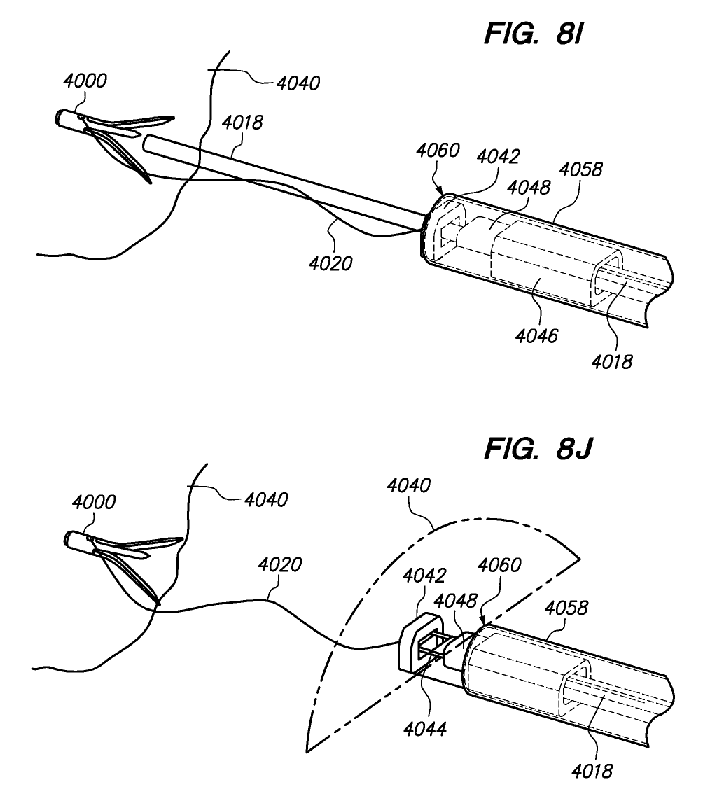

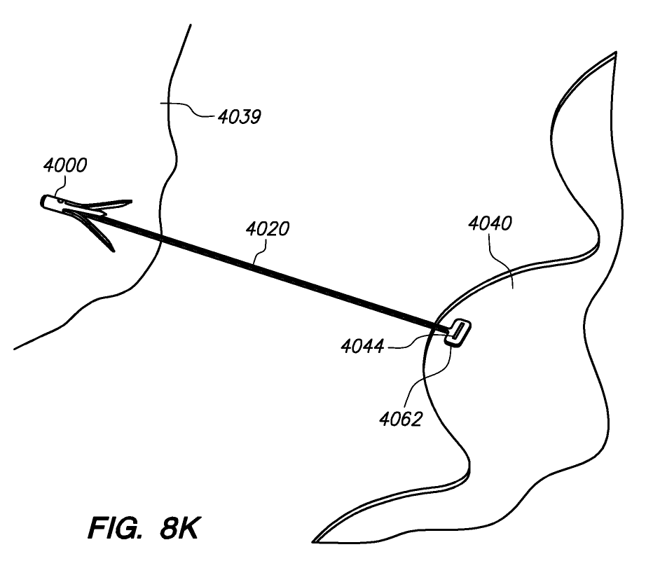

FIG. 4C illustrates another first anchor configuration, termed a dart anchor (4000). The depicted dart anchor (4000) comprises three proximal legs (4012) and a tubular distal body (4014) having an inner diameter (4006), outer diameter (4004), and defining a lumen (4002) therethrough, as depicted in the body (4014) cross sectional illustration of FIG. 4D. A working space is defined in between the plurality of legs (4012). Suitable dimensions for one embodiment of the dart anchor (4000) are illustrated in FIGS. 4C and 4D. Referring to FIG. 4E, two different delivery configurations are depicted. Referring to the leftmost depicted configuration, a sharp-tipped (4026) delivery instrument (4024) defining a delivery lumen (4028) containing a dart anchor (4000), in a compressed form with the legs compressed to form a proximal dart outer shape diameter roughly equivalent to that of the distal body portion of the dart anchor, coupled to the distal end of a dart insertion probe (4017) advanceable relative to the delivery instrument (4024) is depicted being advanced, sharp end (4026) first, into a targeted tissue structure (4030), such as a papillary muscle head. As illustrated immediately to the right of the leftmost assembly, withdrawal of the delivery instrument (4024), and/or insertion of the dart anchor (4000) with the insertion probe (4017), leave the dart anchor (4000) deployed into the tissue structure in its expanded form with a tensile member (4020), such as a suture or PTFE chord, coupling the dart (4000) to the deployment structures, or other prosthesis structures such as a second anchor. The two rightmost assemblies depicted in FIG. 4E illustrate another deployment configuration wherein a sharp distal end (4022) of an insertion probe (4018) is positioned through the dart body lumen and dart legs working space. As shown in FIG. 4E, this configuration may be utilized to directly advance the dart anchor (4000) into the targeted tissue structure (4031) and then controllably detach, leaving behind the dart anchor (4000) and associated tensile member (4020). As the dart anchor (4000) is advanced with this configuration, the legs of the dart anchor may be configured to flex toward the central longitudinal axis of the dart anchor, thereby minimizing insertion trauma, until the dart anchor (4000) has been sufficiently advanced, after which the legs may be configured to expand outward to prevent migration of the dart anchor (4000), particularly in reaction to tensile forces which may be applied to the dart anchor (4000) through the tensile member (4020). The sharp tip (4022) of the insertion probe (4018) placed through the lumen of the dart anchor body, along with a distal tapering shape (4016 in FIG. 4C) of the dart anchor body, also assist with relatively atraumatic advancement and insertion through tissues, such as viscoelastic tissues, during delivery. A relatively high impulse insertion load/timing profile over a predetermined insertion deflection may be utilized to advance the dart anchor (4000) into position. The dart anchor (4000) and delivery insertion probes (4017, 4018) may be controllably detachable using mechanical interfaces, such as mechanically threaded interfaces detachable with relative rotational motion, electromagnetically controllable detachment interfaces, whereby a current or potential difference may be configured to cause detachment, or by controlled erodible link techniques, as described, for example, in U.S. Pat. No. 5,122,136, which is incorporated by reference herein in its entirety. Referring to FIG. 4F, a close up view of a composite delivery instrument (4032) defining two working lumens to accommodate independently controlled activity of a dart anchor (4000) and insertion probe (4018) through a first working lumen (4034), and independent activity of a steerable grasping instrument (4038) through a second working lumen (4036) may be utilized to grasp a targeted tissue structure and, for example, provide traction/countertraction forces relative to the insertion forces which may be applied to advance the dart anchor (4000) into position in such targeted tissue structure. Assemblies such as those featured in FIG. 4E, and FIGS. 4F and 8B-8K described in further detail below, may be navigated to targeted tissue structures using the working lumens of devices such as cannulas or remotely steerable catheters, such as those that are manually steerable utilizing proximal steering handle assemblies, or those that are electromechanically steerable, such as the robotic catheter systems available under the tradename Sensei® by Hansen Medical, Inc., of Mountain View, Calif. Alternatively, navigation or steerability components, such as steering pullwires and pullwire terminations, may be integrated directly into the insertion probes.

FIG. 5 simply demonstrates the first anchor (402) being deployed into the head of papillary muscle (304) by distal advancement of the plunger or piston-like mechanism (404). After the first anchor (402) is fully deployed into the head of papillary muscle (304), its legs may expand to further secure the anchor into the cardiac tissue, as shown in the variation of FIG. 4B. Cord (400) may be pulled proximally to ensure first anchor (402) is firmly and adequately secured to the head of papillary muscle (304) or other cardiac tissue as the case may be. If the anchor is not firmly secured, the deployment mechanism (e.g., hydraulic, plunger, or piston (404)) may be retracted proximally, the anchor withdrawn, and then re-deployed by the deployment mechanism (e.g., by distal advancement of a plunger or piston (404)).

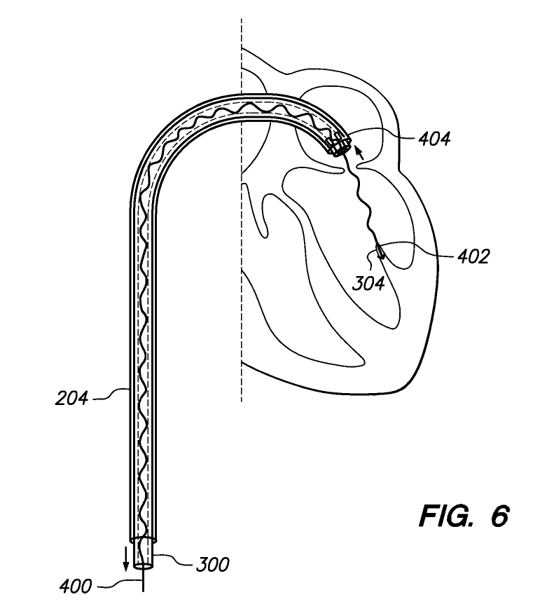

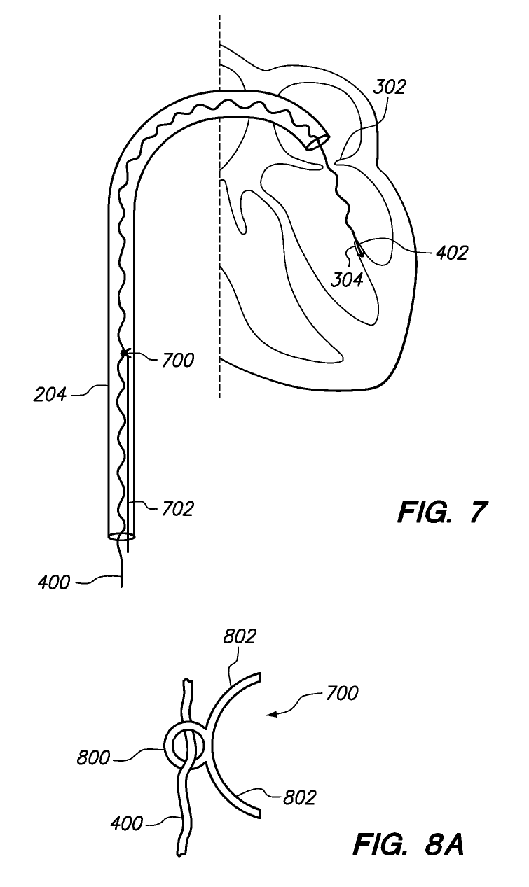

After the first anchor (402) has been firmly secured to cardiac tissue located beneath the prolapsed mitral valve (e.g., into the head of the papillary muscle, 304), the suction to catheter (300) is released, and catheter (300) is withdrawn proximally (shown by arrows), leaving the first anchor (402) attached to cord (400) in place, as shown by FIG. 6. FIG. 7 provides an illustrative depiction of advancement of a leaflet, or “second” for the purposes of the embodiments illustrated in FIGS. 1-16C (and “leaflet anchor (700)” thereafter) anchor (700) along cord (400). Second anchor (700) is slidably attached to the cord (400) and is configured to secure into the prolapsed mitral valve leaflet. The second anchor is typically slid onto the cord (400) proximal of the patient's body, and introduced and advanced to the site of the prolapsed mitral valve with the aid of a catheter (702). In FIGS. 7, 9 and 10, the catheter introducing such anchor is indicated by a line (702). Any appropriate introducer for positioning, releasing and securing the anchor may be used for example, the second anchor may be positioned and released as described above for the first anchor. A practitioner may thread the second anchor onto the cord outside of the body. After the second anchor has been attached to the prolapsed mitral valve leaflet (302), the valve leaflet may be repositioned to correct the prolapse, and tension may be applied to the cord (400) to maintain the proper position of the anchors to correct the prolapse.

FIG. 8A shows an illustrative depiction of a second anchor (700) in a magnified fashion. Shown there is anchor (700) slidably attached to cord (400). In this variation, second anchor (700) has eyelet (800) allowing for such slidable attachment (i.e., in this variation, the cord is slid through eyelet 800). The second anchor of this variation also has legs (802) that are configured to pierce tissue, and preferably, the tissue of a prolapsed mitral valve leaflet. Again, the legs of the second anchor need not terminate in sharp or otherwise pointed distal tips, so long as the anchor has been suitably designed to pierce tissue. Similarly, as with the first anchor described in detail above, the second anchor may be made of any suitable material, and be of a size suitable to secure into a prolapsed mitral valve leaflet. For example, in some variations the anchor is between about 4 mm and about 8 mm (e.g., the tips of the legs span between about 4 mm and about 8 mm). In some variations, the anchor is about 6 mm.

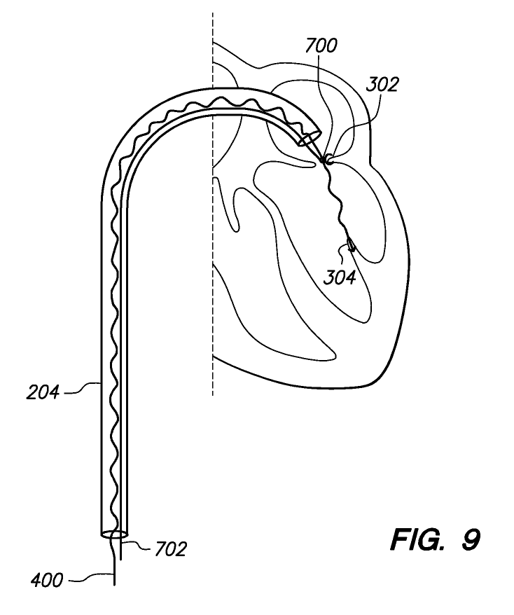

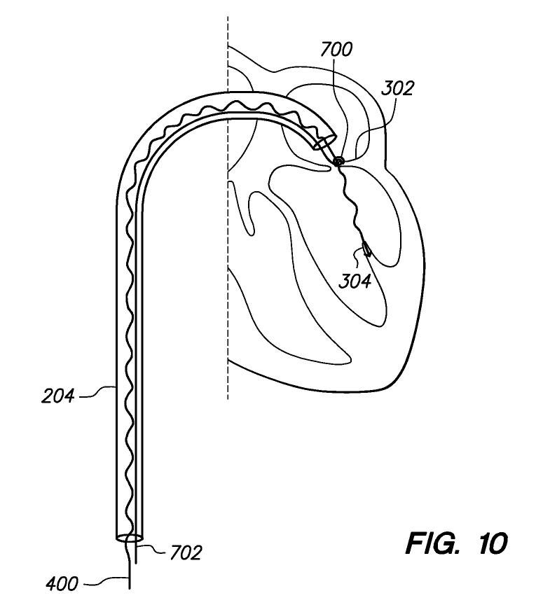

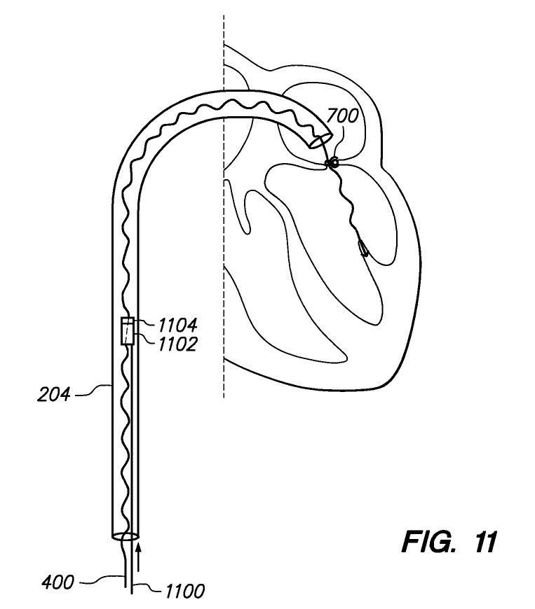

As noted above, the second anchor (700) may be advanced to prolapsed mitral valve leaflet (302) via catheter (702). Catheter (702) is typically steerable, or otherwise maneuverable so that it can be maneuvered through trans-septal catheter (204) and to the prolapsed mitral valve leaflet (302). Visualization techniques, such as fluoroscopy may be useful in this respect. The second anchor may be attached to the catheter, and released only after it has been attached to the valve leaflet. In some variations, the second anchor may pierce (e.g., pass completely through) the valve leaflet when the second anchor is secured to the valve leaflet. For example, the second anchor may be positioned by abutting the valve leaflet with one or more of the anchor legs, so that the practitioner can cause the anchor legs to penetrate into the valve leaflet. The anchor may be secured to the margin (e.g., within about 5 mm from the edge of the leaflet) or in any other appropriate region of the valve leaflet. In the variation shown in FIG. 9, the legs of anchor (700) are positioned so that they may be inserted into the valve leaflet. For example, the legs may straddle the edge of prolapsed mitral valve leaflet, or one of the legs may hook onto the mitral valve leaflet (302). The second anchor (700) should preferably be positioned at the edge of the mitral valve leaflet, but the legs of the anchor may secure into a deeper margin of the prolapsed mitral valve leaflet if desirable. In some variations, it may be preferable for the practitioner to hook the lower edge of the prolapsed mitral valve leaflet with the lower leg of the second anchor, before the legs are collapsed or expanded (as the case may be) into the leaflet tissue.

FIG. 8B illustrates a compact stapler assembly (4054) which may be utilized to deploy a second anchor into a tissue structure when a portion of such tissue structure may be fitted into the jaw space (4056) defined by the assembly between a distal anvil structure (4042), and one of the members defining the proximal jaw space, including a housing (4046), advanceable jaw member (4048), and advanceable stapling member (4050). The advanceable jaw member (4048) and advanceable stapling member (4050) may be advanced relative to the housing (4046) and/or the delivery instrument (4052) from a proximal operator position wherein an operator may manipulate proximal portions of these members (4048, 4050) or other members coupled thereto. Referring to FIG. 8C, in operation, a tissue structure (4040), such as a mitral valve leaflet, is placed into the jaw space (4056). Referring to FIG. 8D, the jaw member (4048) may be advanced to hold a portion of the tissue structure (4040) in compression against the anvil portion (4042). Referring to FIG. 8E, the stapling member (4050) may be advanced toward the anvil (4042) to drive a staple (4044) through the captured tissue structure portion, to the anvil (4044), and bent back around back into the captured tissue structure portion, leaving behind a deployed staple (4044), as illustrated in FIG. 8F. Also shown in FIG. 8F is a staple (4045) deployed in a different orientation, to illustrate that the assembly may be rolled relative to its longitudinal axis to place staples at various orientations. Referring to FIGS. 8G-8K, a single delivery instrument (4058) assembly may be utilized to deploy both first and second (or distal and proximal) anchors, with a tensile member, such as a suture, in between. As shown in FIG. 8G, a dart anchor (4000) and deployment probe (4018) may be inserted through a lumen defined through a compact stapling assembly similar to that depicted in FIG. 8B, which exception that a working lumen is defined therethrough to accommodate relative motion of the dart anchor (4000) and delivery insertion probe (4018). Referring to FIG. 8H, when the delivery instrument (4058) has been advanced and positioned adjacent a desired tissue structure, such as a papillary muscle, the dart anchor delivery insertion probe (4018) may be advanced out and used to deploy a dart anchor (4000) and untensioned tensile member (4020), as depicted in FIG. 8I. Referring to FIG. 8J, the dart anchor delivery insertion probe (4018) may be withdrawn and the delivery instrument (4058) may be utilized to position the jaw space of the compact stapling assembly in position to deploy a staple (4044) into a targeted tissue structure (4040), such as a mitral valve leaflet, as described in reference to FIGS. 8B-8F above, the staple (4044) remaining coupled to the tensile member (4020), and thereby coupled to the dart anchor (4000). Referring to FIG. 8K, the tensile member (4020) may be tensioned and clipped, using techniques such as those described above. In one embodiment, the compact stapler may be configured to also deploy an implantable pledget (4062) along with the staple (4044) to spread loads and provide a more optimal coupling interface, as depicted in FIG. 8K.

FIG. 10 provides an illustrative depiction of the second anchor (700) deployed into the prolapsed mitral valve leaflet (302). The deployment of second anchor (700) may occur in any number of ways. For example, a release mechanism may be activated from outside the patient's body. For example, the release mechanism may comprise a reversibly releasable latch within catheter (702). Having a reversible latch, for example, may be quite beneficial so that the practitioner may have multiple tries at deploying the second anchor, if the first try results in misplacement. The release mechanism, may be made of a cable with a releasable latch at its distal end, for example, or something similar. As described above, the legs of the anchor may be deployed into the tissue by changing the configuration of a shape-memory material or by releasing from a loading configuration (e.g., compressed) into a delivery (e.g., expanded) configuration.

As described above, the legs of the second anchor can be secured into the prolapsed valve leaflet by any suitable mechanism. For example, the second anchor may be made from a self-expanding material, such as a shape memory material (e.g., a nickel titanium alloy), such that, when it is released, its legs expand (or compress or collapse as the case may be) into the tissue of the mitral valve leaflet. Similarly, an expandable balloon may be used to force the legs of the anchor into the prolapsed mitral valve leaflet tissue.

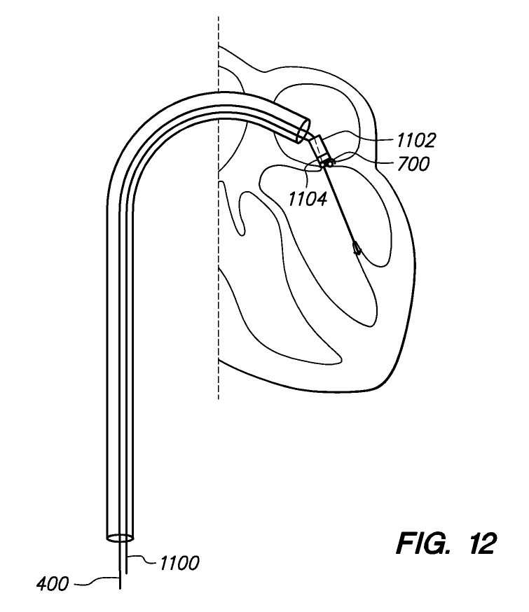

FIG. 11 shows a schematic representation of a catheter (1100) having a releasable fastener (1104) at its distal end (1102) being advanced distally through trans-septal catheter (204). As shown, the distal end (1102) and releasable fastener (1104) have lumens therethrough, which allow them to be thread or slid over cord (400). Catheter (1100) is advanced distally as shown by the arrows, and will be advanced until the releasable fastener (1104) is positioned immediately adjacent to the second anchor (700) as shown by FIG. 12. The position of the valve leaflet is then adjusted (e.g., to correct the prolapse and eliminate mitral regurgitation). Thus, the fastener (1104) may be used to position the valve leaflet. For example, the fastener may be used to push against the second anchor (700) (e.g., by pushing against the eyehole of the fastener). At the same time, the cord (400) can be tensioned to correct the position of the valve leaflet. This position can be maintained by securing the cord (e.g., with the fastener (1104)). A practitioner may adjust the position of the valve leaflet (e.g., by adjusting the position of the fastener and the tension of the cord) while visualizing the heart to correct a prolapse. Correction is obtained when regurgitation into the left atrium disappears or is significantly reduced. The correction can be monitored, for example, by intracardiac echo or transesophageal echo. Thus, the position of the valve leaflet can be adjusted in real time while monitoring any regurgitation. Once the position is optimized, the fastener (1104) may be secured.

As described above, the releasable fastener may be positioned to optimally tension the cord (400) so that the distance between the anchors adequately corrects the position of the prolapsed leaflet. The releasable fastener is then released and fastened or crimped to the cord (400) to secure the cord in this position.

The prolapsed leaflet may be repositioned to correct the prolapse by the practitioner manipulating the valve leaflet, and by applying tension (e.g., pulling) on the cord. In some variations, the practitioner may use a separate manipulator to move the valve leaflet and to secure the cord position. For example, a wire or another catheter (e.g., separate from the catheter used to deliver the fastener) may be used to push against the leaflet or anchor.

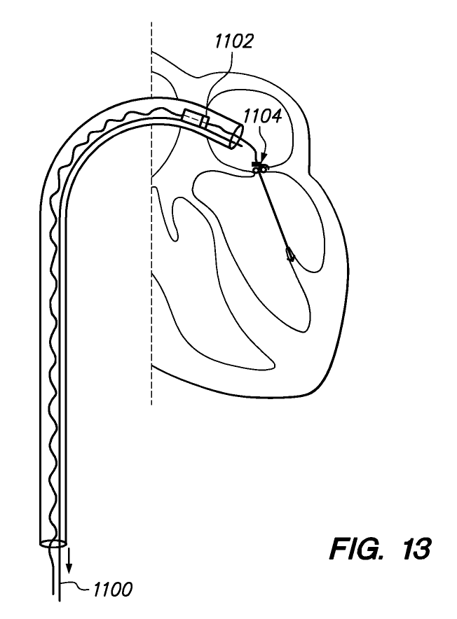

The fastener may be released and secured (e.g., crimped) in any suitable way. For example, the fastener may be held in catheter (1102) by a sheath, and once the sheath is removed, the fastener may be released. Similarly, the fastener may be attached to the catheter by an electrolytic joint, and once it is desired to detach the fastener, electricity may be applied to sever the joint via electrolysis. The fastener may also be released via the use of a push-pull wire. Any number of suitable techniques may also be used with respect to crimping of the fastener. For example, the fastener may be made of a self-collapsing material, such as a shape memory material, that would collapse tightly upon itself after being released. An expandable balloon may also be used to crimp the fastener. That is, as the expandable balloon is inflated, the fastener is crimped tightly upon itself under the pressure. After the fastener is positioned and crimped in place, catheter (1102) is withdrawn proximally as shown by FIG. 13.

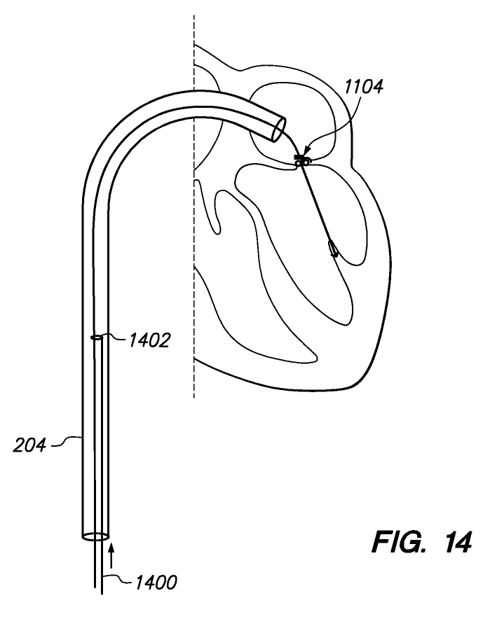

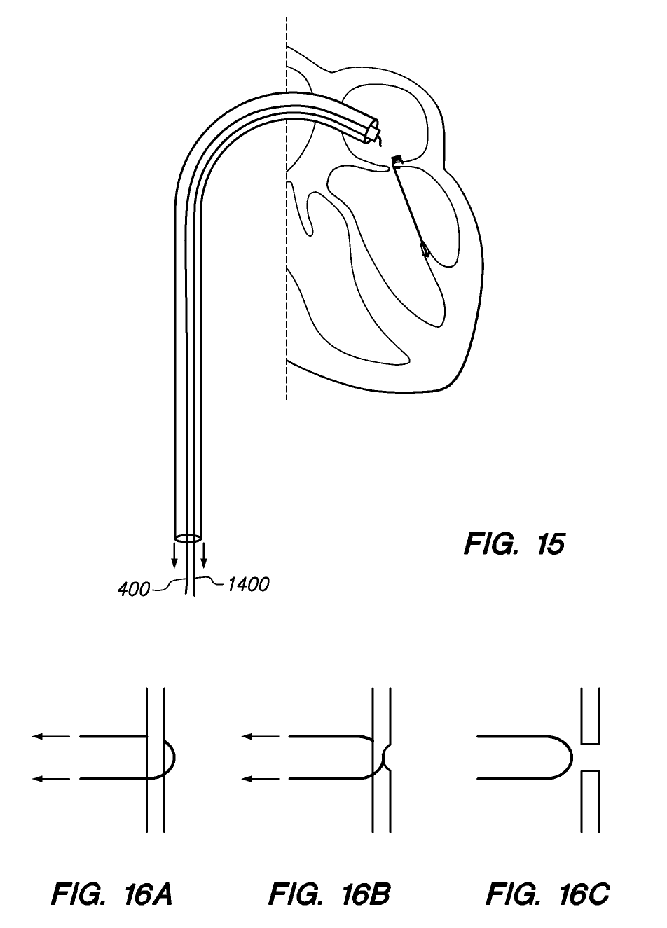

After the prolapse has been corrected via the tightening and securing of the cord, the excess cord remaining in the heart must be cut and removed from the body. This may be accomplished in any number of ways. For example, a cutting catheter (1400) may be advanced distally through trans-septal sheath (204) as shown in. FIG. 14. As shown in that figure, cutting catheter (1400) has a cutting mechanism (1402) at its distal end. The cutting mechanism may be any suitable mechanism, and in the variation shown in FIG. 14, it is a cutting wire having a loop. In this variation, the looped cutting wire is slid over the cord (400) and advanced immediately proximal to the fastener (1104). The looped cutting wire may be made of any suitable biocompatible cutting material, e.g., stainless steel and the like. FIGS. 16A-16C illustrate a cutting wire cutting a cord. The excess cord is cut off, and both the excess cord (400) and the cutting catheter (1400) are removed proximally as shown by FIG. 15. The cord may also be cut at the same time that the fastener is secured and/or released. For example, the same device that deploys the fastener may also cut the cord. In some variations, the cord is cut at the same time that the fastener is secured and/or released.

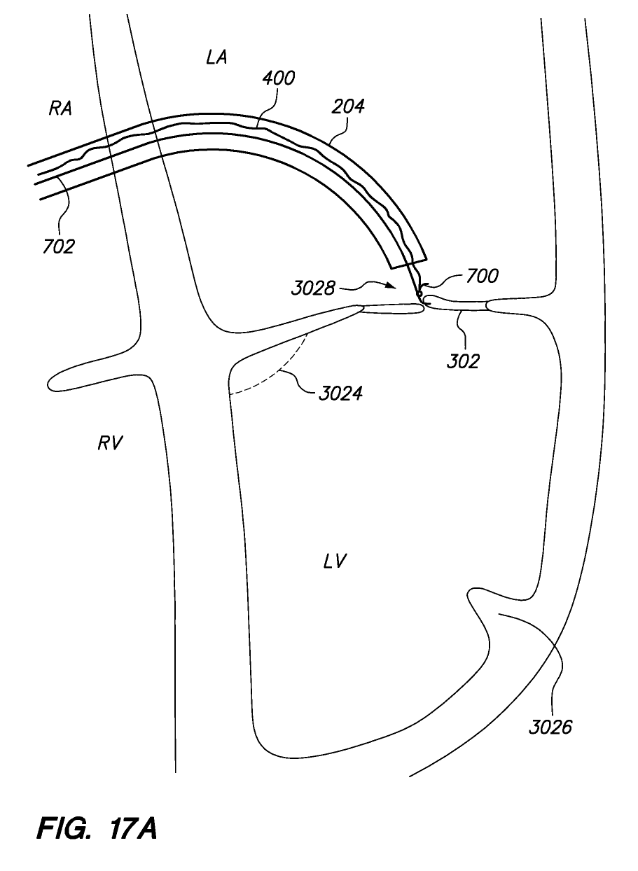

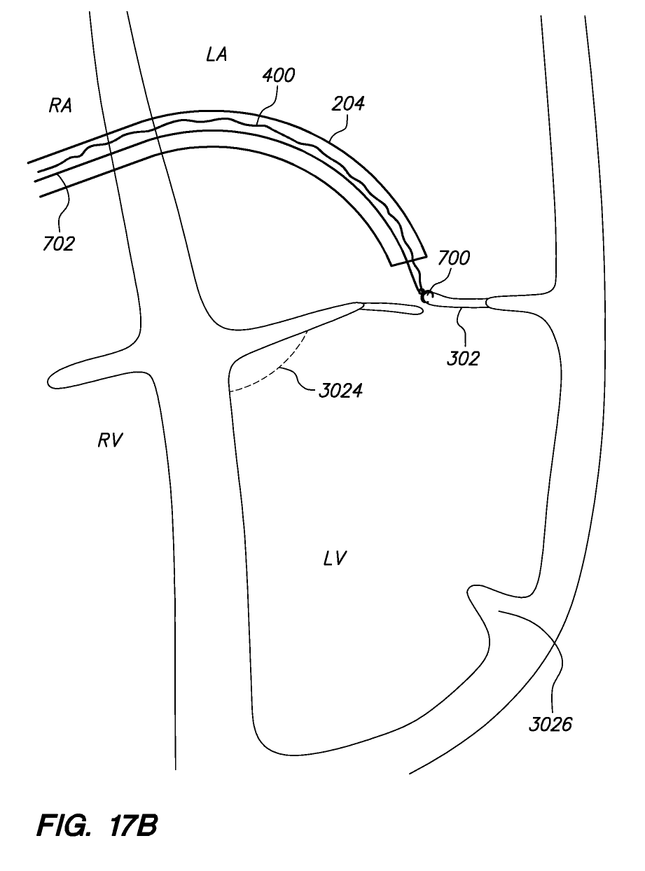

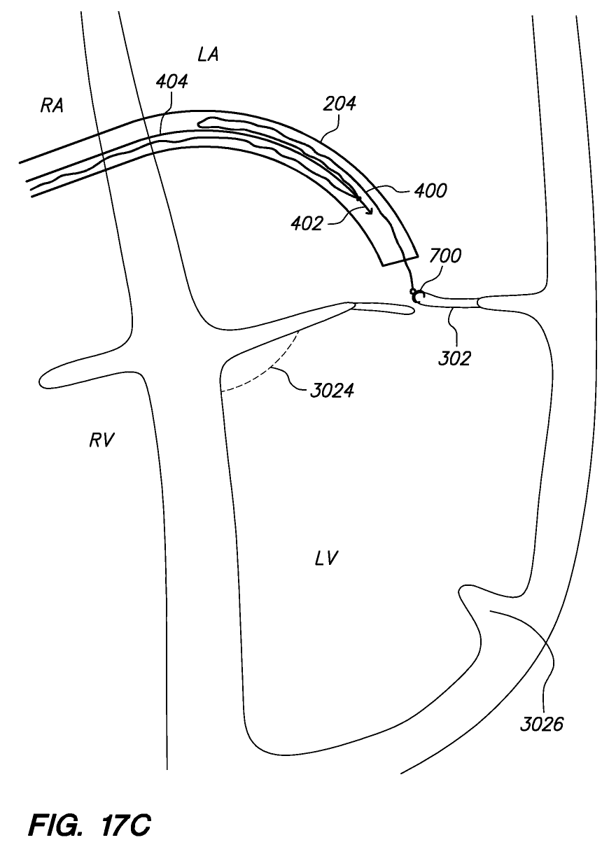

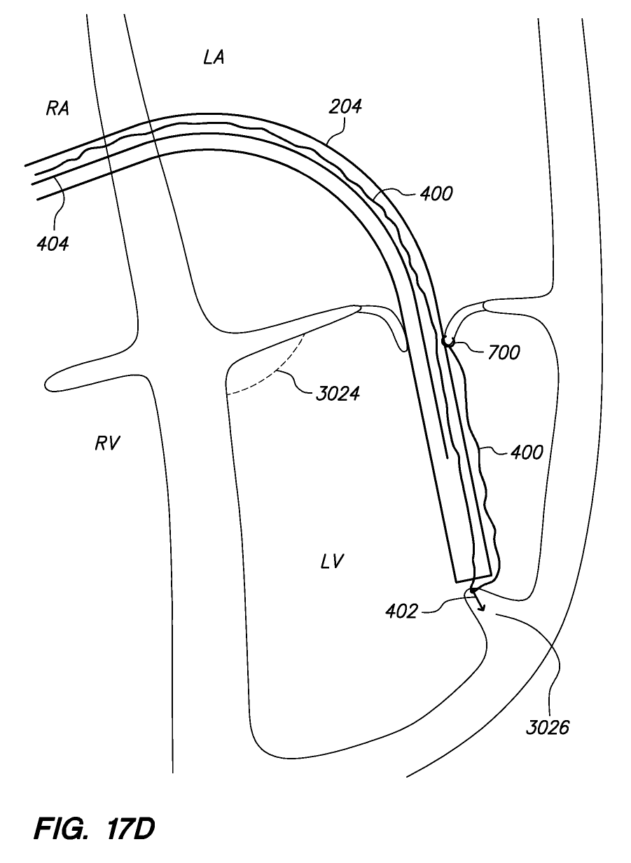

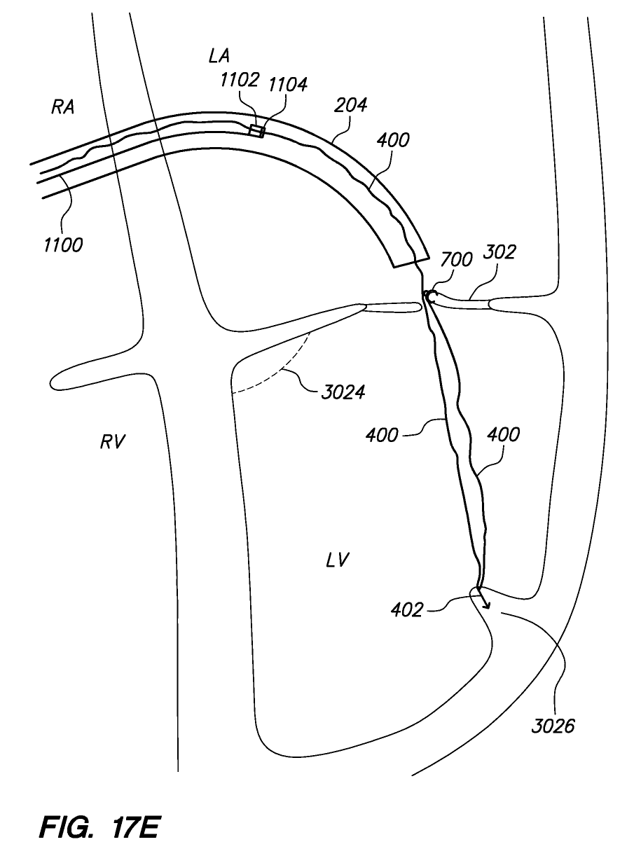

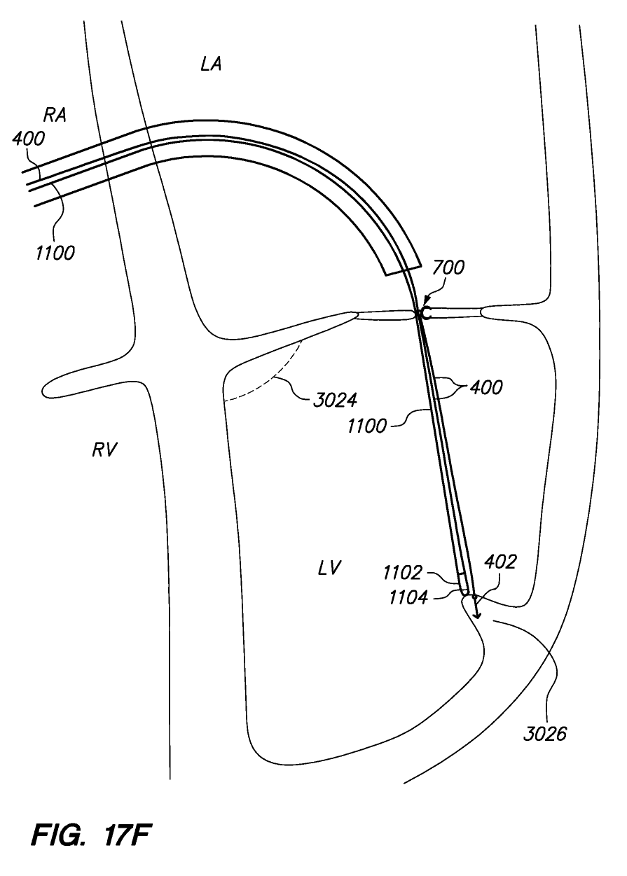

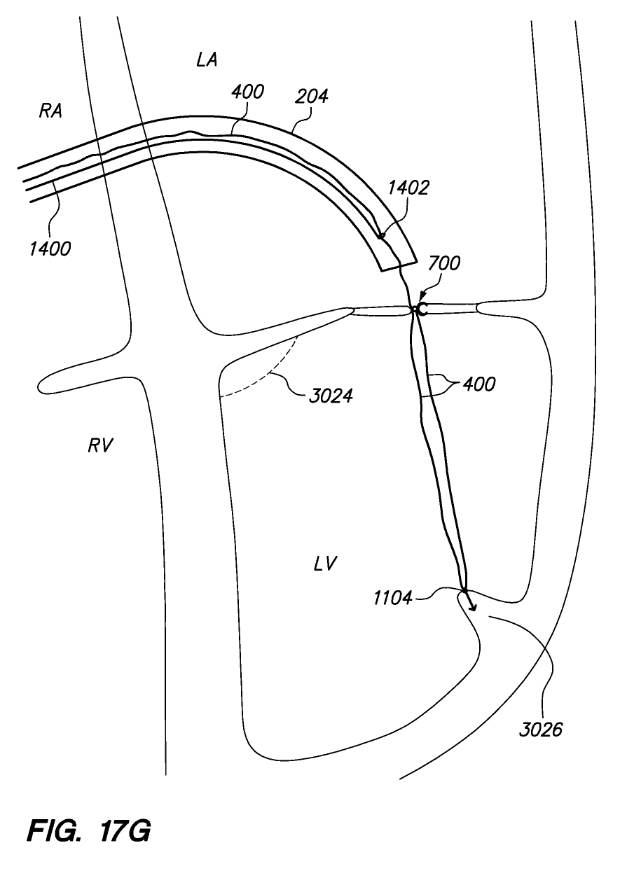

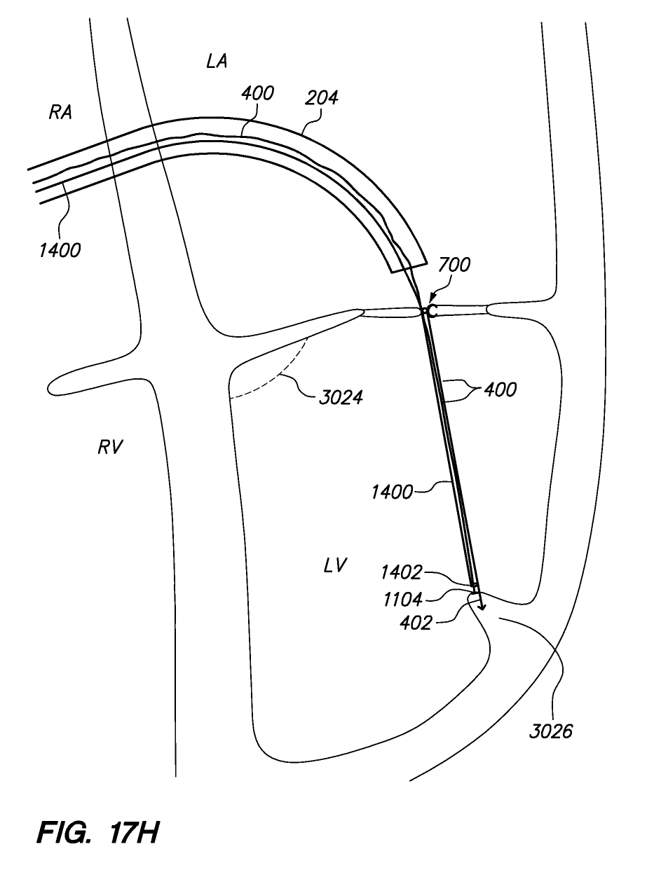

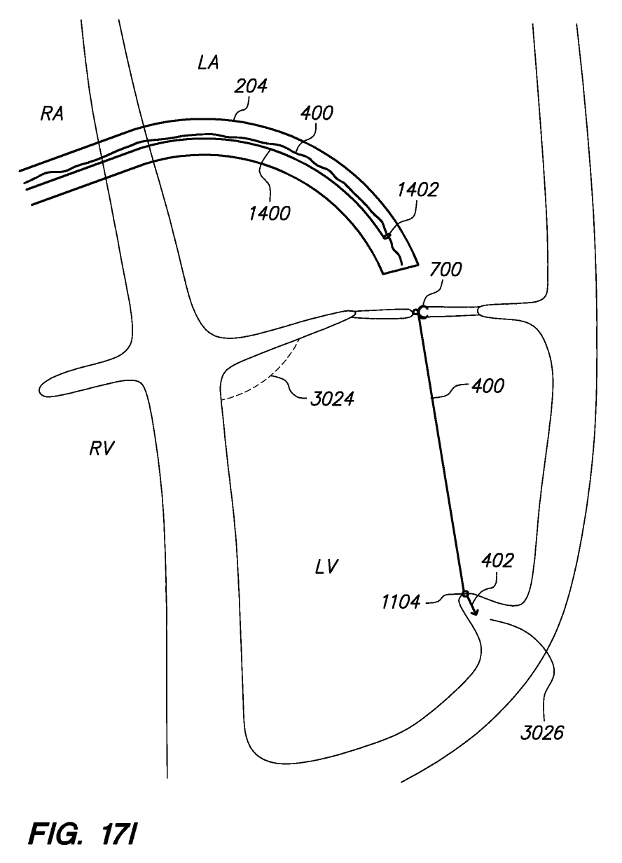

Referring to FIGS. 17A-27C, additional embodiments of the inventive system, method, and apparatus are illustrated utilizing many of the components described in reference to FIGS. 1-16C.

FIGS. 17A-17I depict a variation wherein an antegrate approach is utilized to deploy a first anchor in a leaflet, followed by deployment of a second anchor in the papillary muscle. Referring to FIG. 17A, a trans-septal catheter (204) is utilized to approach the mitral valve (3028). A leaflet anchor (700) introducer catheter (702) is utilized to position the anchor (700) against the desired leaflet margin on the prolapsed mitral leaflet (302). Referring to FIG. 17B, the leaflet anchor (700) is fastened to the leaflet utilizing a mechanism as described in reference to FIG. 10. Referring to FIG. 17C, with the leaflet anchor (700) deployed and the corresponding introducer catheter (702 in FIG. 17B) removed, a papillary anchor (402) is advanced with an introducer, such as a plunger or pistonlike mechanism, (404) toward the end of the transseptal catheter (204) with a cord (400) coupled to both anchors (402, 700) and leading proximally to an extracorporeal location accessible by the surgeon. Referring to FIG. 17D, the transseptal catheter (204) is advanced across the mitral valve (3028) toward the papillary muscle (3026), the papillary anchor (402) is deployed, and the introducer (404) is withdrawn. Referring to FIG. 17E, a fastener catheter (1100) distal end (1102) having a releasable fastener (1104) is advanced over the cord (400) material, and in FIG. 17F, across the mitral valve (3028) and down to the location of the deployed papillary anchor (402). The cord (400) may be tensioned from an extracorporeal location to optimize valve mechanics before the releasable fastener (1104) is deployed and left in place, as depicted in FIG. 17G. Referring again to FIG. 17G, and also to FIG. 17H, a cutting catheter with cutting mechanism (1402) is advanced toward the fastener (1104). Referring to FIG. 17I, with the cord (400) length optimized (i.e., to prevent prolapsed of the engaged mitral valve leaflet), the cutting mechanism (1402) has been utilized to release the cord (400) and fastener (1104), leaving the prosthetic chordae tendinae configuration in place, and the remaining interventional devices (204, 1400, 1402, proximal remnants of the cord 400) may be removed proximally.

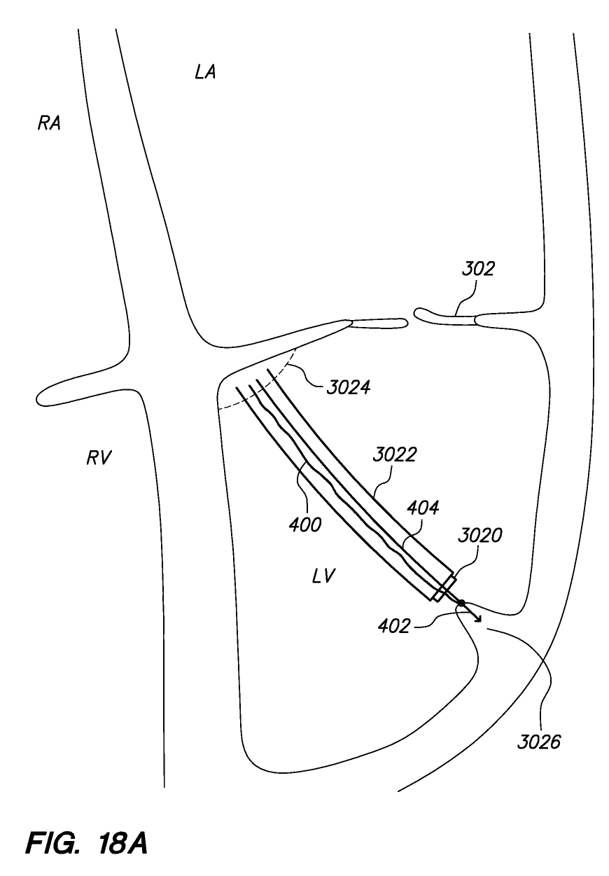

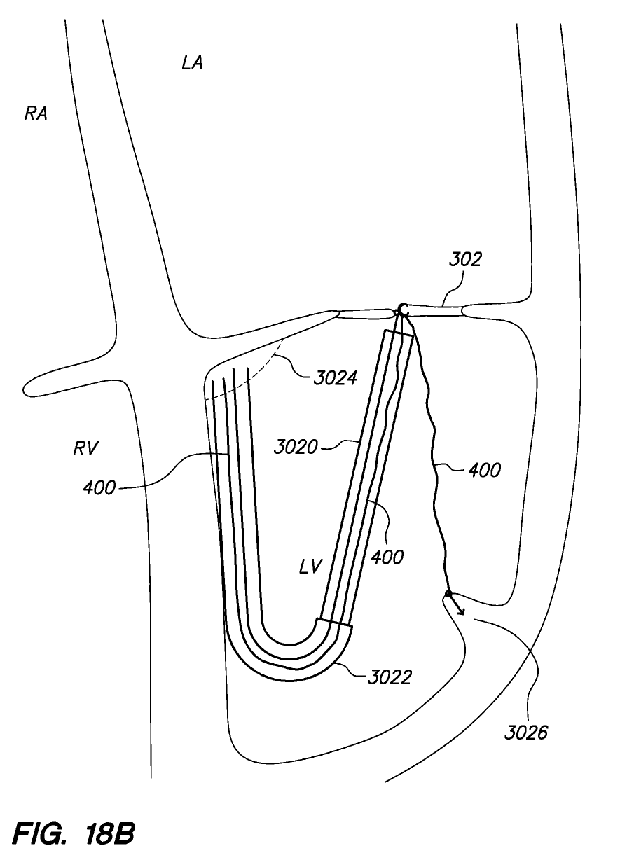

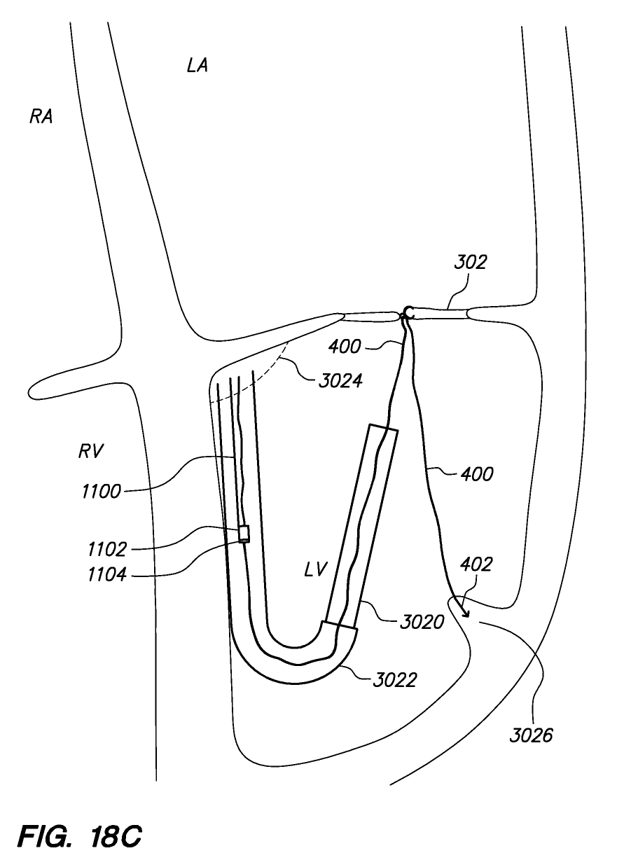

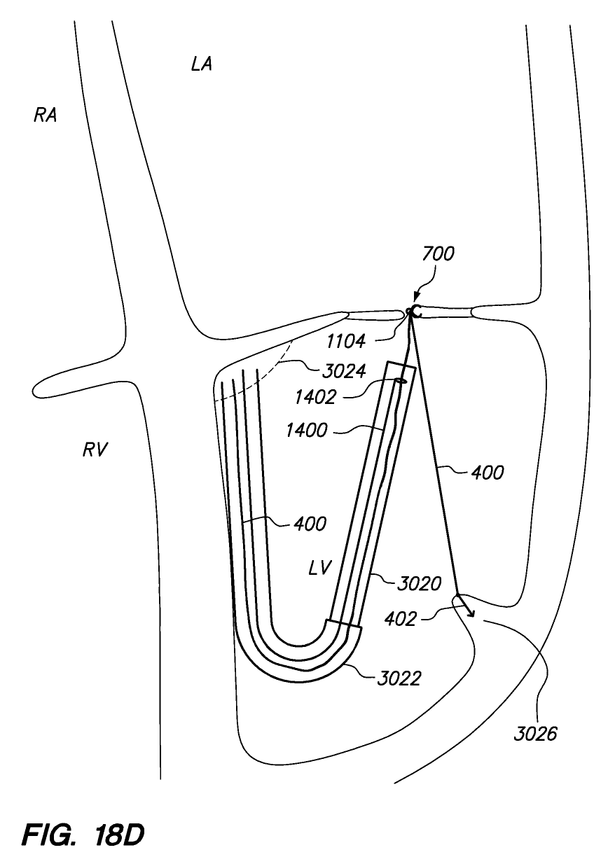

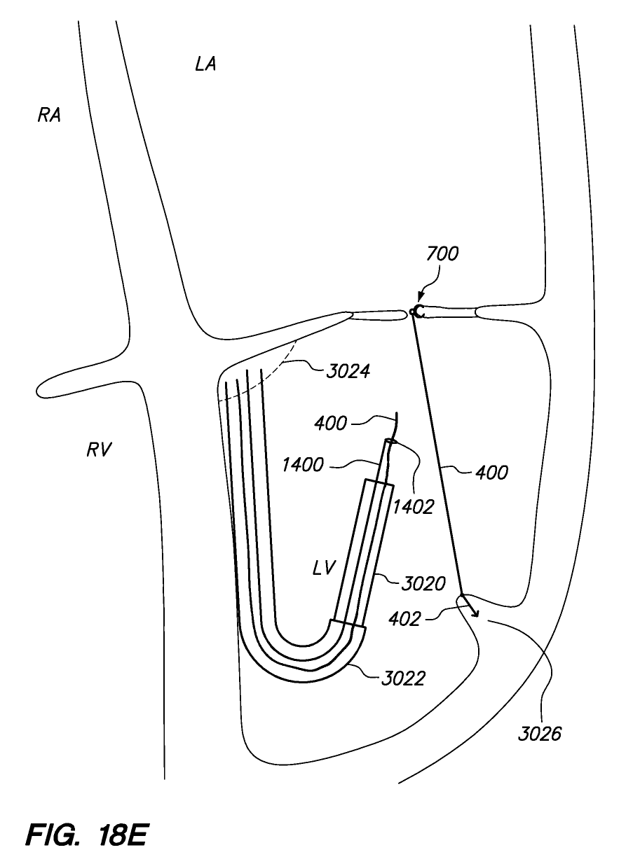

Referring to FIGS. 18A-18E, an embodiment is depicted wherein a trans-aortic, retrograde approach is utilized to deploy a papillary anchor (402) first, followed by a leaflet anchor (700). Referring to FIG. 18A, a working catheter complex, which may, for example, comprise two coaxially-coupled catheters (an inner catheter 3020, and an outer catheter 3022) optimized to provide a range of motion throughout distal regions of the left ventricle, is depicted. In other variations, a more conventional single steerable catheter, probe, or other flexible instrument, or combination thereof, may be utilized to navigate the left ventricle and provide retrograde access to pertinent tissue structures. In the depicted embodiment, the inner (3020) and outer (3022) catheters may be operated from an extracorporeal location to insert and roll relative to each other and the patient, and preferably steer relative to each other. Referring again to FIG. 18A, the catheter complex is advanced across the aortic valve orifice (3024) and a papillary anchor (402) is deployed into the papillary muscle (3026). Subsequently the papillary anchor introducer (404) may be withdrawn proximally. Referring to FIG. 18B, the catheter complex (3020, 3022) is repositioned to facilitate deployment of a leaflet anchor (700) with an introducer (702). Referring to FIG. 18C, with the leaflet anchor (700) deployed, the cord (400) may be maintained in slack, running proximal to an extracorporeal location, and utilized to advance a fastener catheter (1100) having a distal end (1102) removably coupled to a fastener (1104). The cord (400) may be tensioned from an extracorporeal location and the fastener (1104) deployed to optimize valve mechanics (i.e., mitigate prolapsed), leaving a configuration such as that depicted in FIG. 18D. Also depicted in FIG. 18D, a cutting catheter (1400) with cutting mechanism (1402) positioned distally is advanced toward the deployed fastener (1104) to facilitate release of the proximal remnant of the cord (400) and removal of the interventional deployment hardware, as depicted in FIG. 18E.

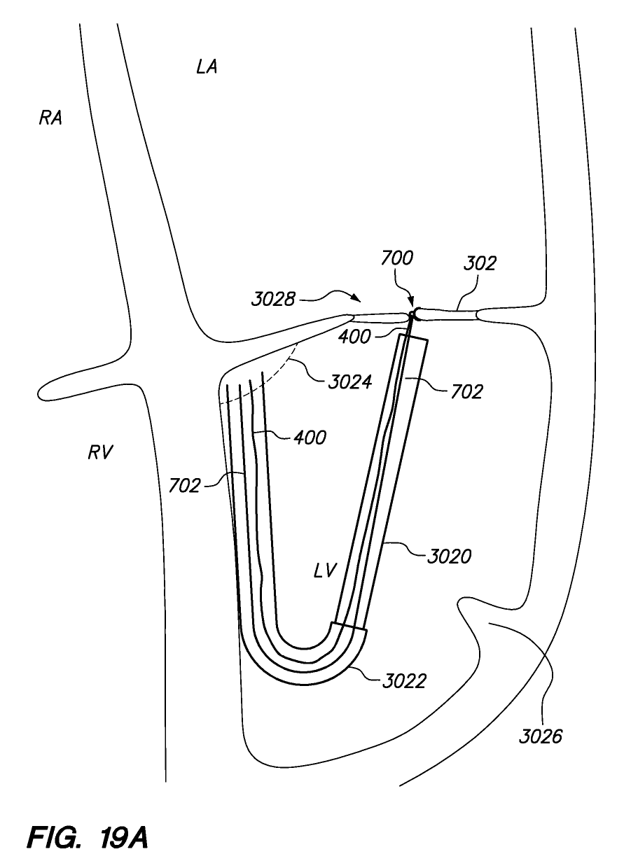

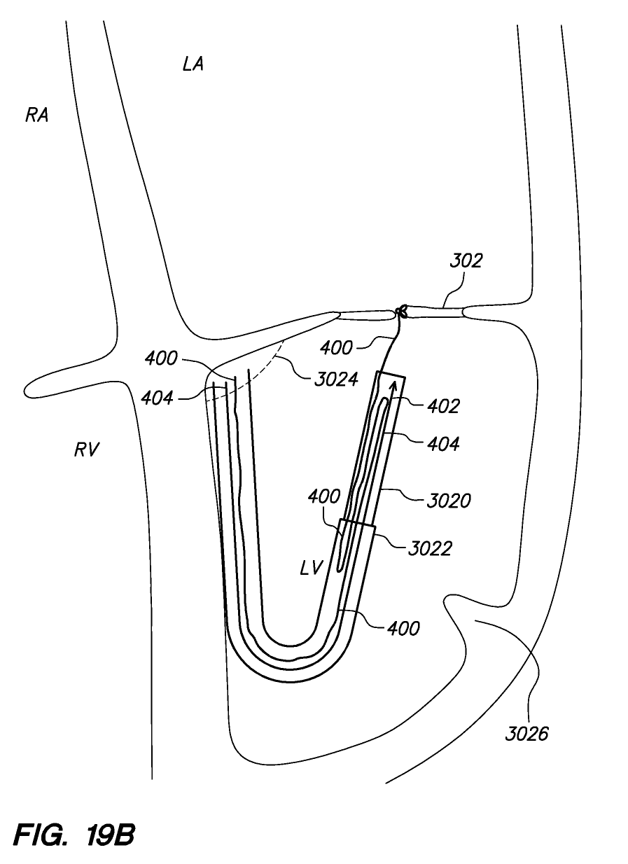

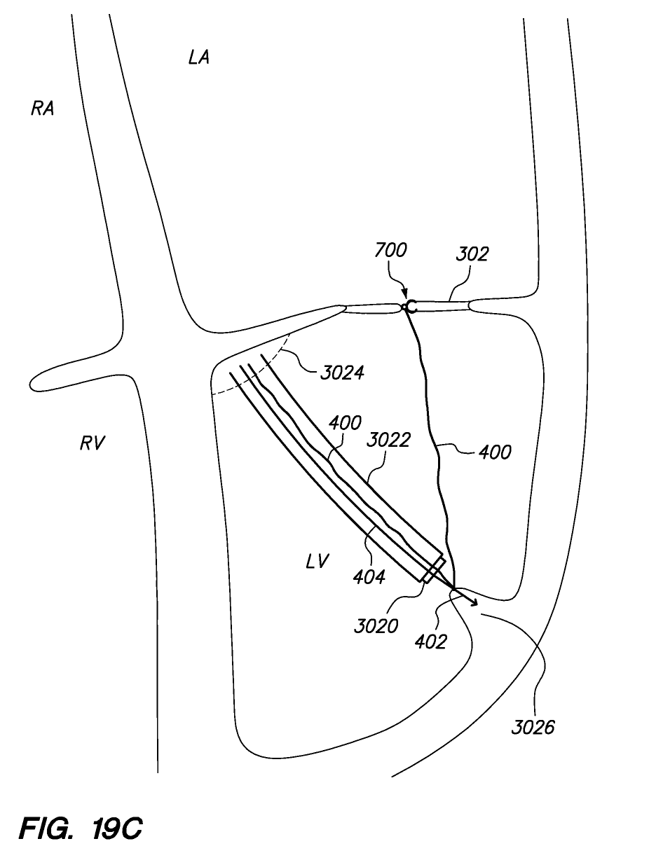

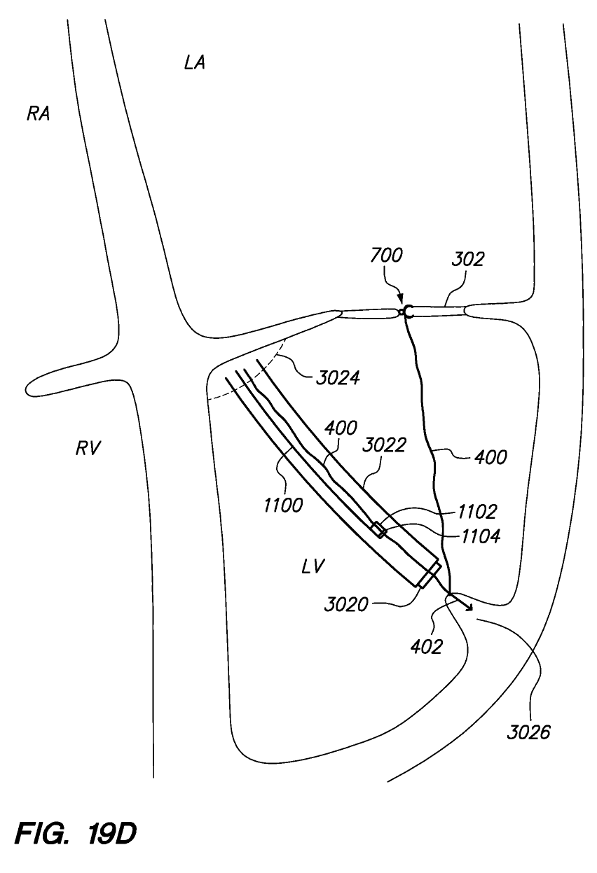

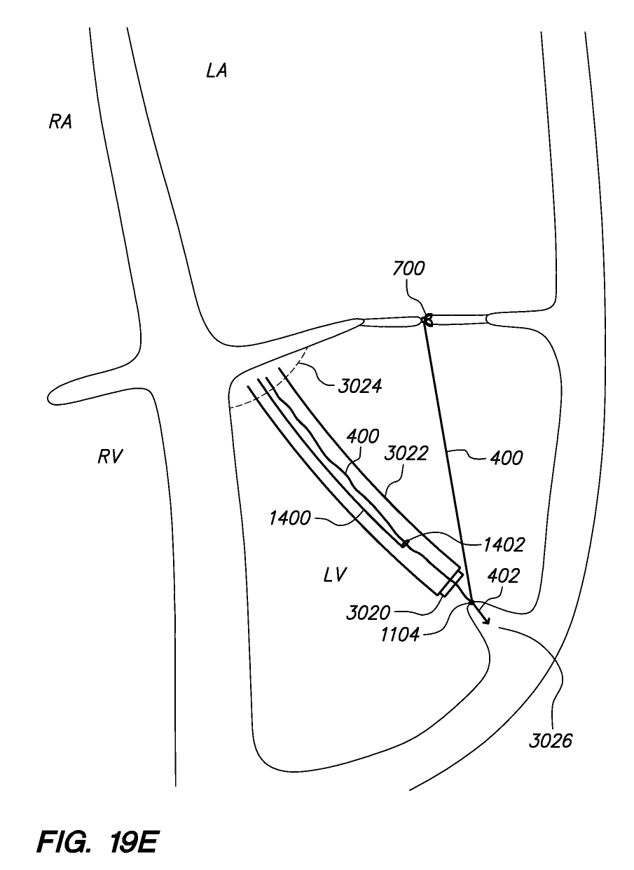

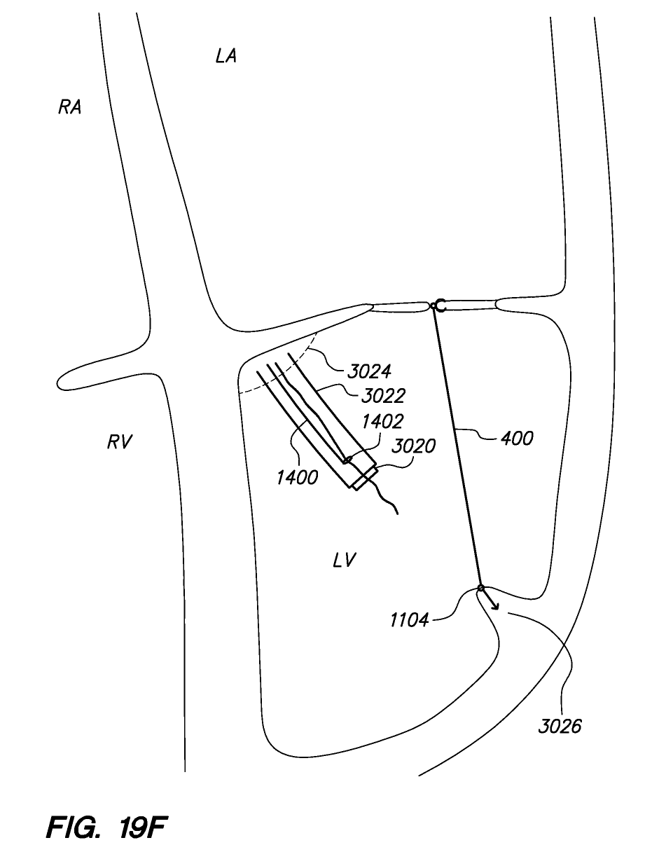

Referring to FIGS. 19A-19F, a retrograde approach is depicted wherein a leaflet anchor (700) is deployed first, followed by a papillary anchor (402). Referring to FIG. 19A, a catheter complex (3020, 3022) is utilized to navigate a leaflet anchor (700) into position along the desired margin of the prolapsed mitral leaflet (302). Referring to FIG. 19B, the introducer (702 in FIG. 19A) has been withdrawn and a papillary anchor (402) is being advanced by an introducer (404) toward the distal end of the inner delivery catheter (3020). Referring to FIG. 19C, the delivery catheter complex (3020, 3022) is moved to facilitate deployment of the papillary anchor (402), after which the papillary anchor introducer (404) may be withdrawn and a fastener catheter (1100) advanced, as depicted in FIG. 19D. The distal portion (1102) of the fastener catheter (1100) may be utilized to release the fastener (1104), subsequent to which a cutting catheter (1400) with distal cutting mechanism (1402) may be advanced, as depicted in FIG. 19E, and utilized to release the proximal cord remnant (400). After release, the delivery hardware may be withdrawn proximally, leaving the deployed anchors (700, 402) and cord (400) section preventing mitral prolapse, as depicted in FIG. 19F.

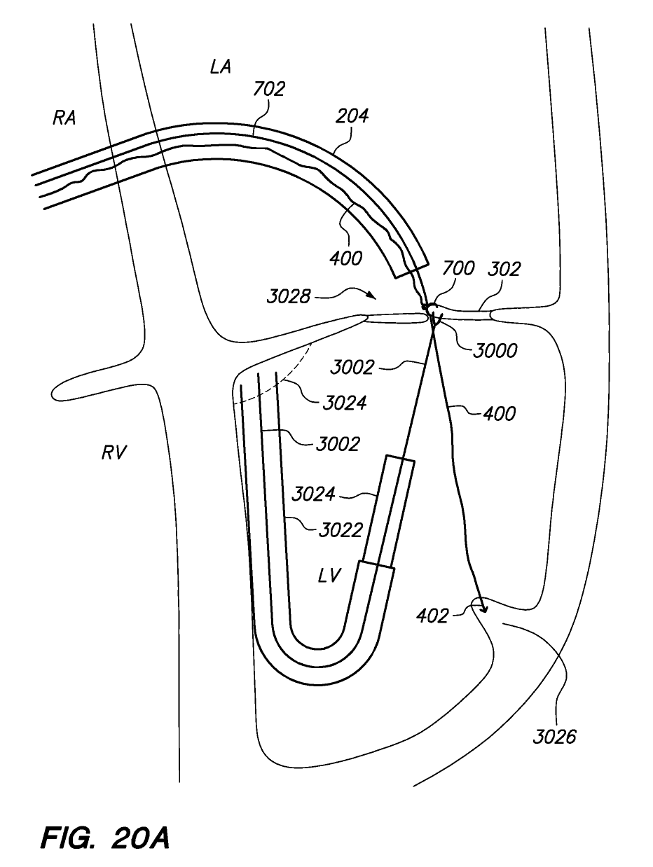

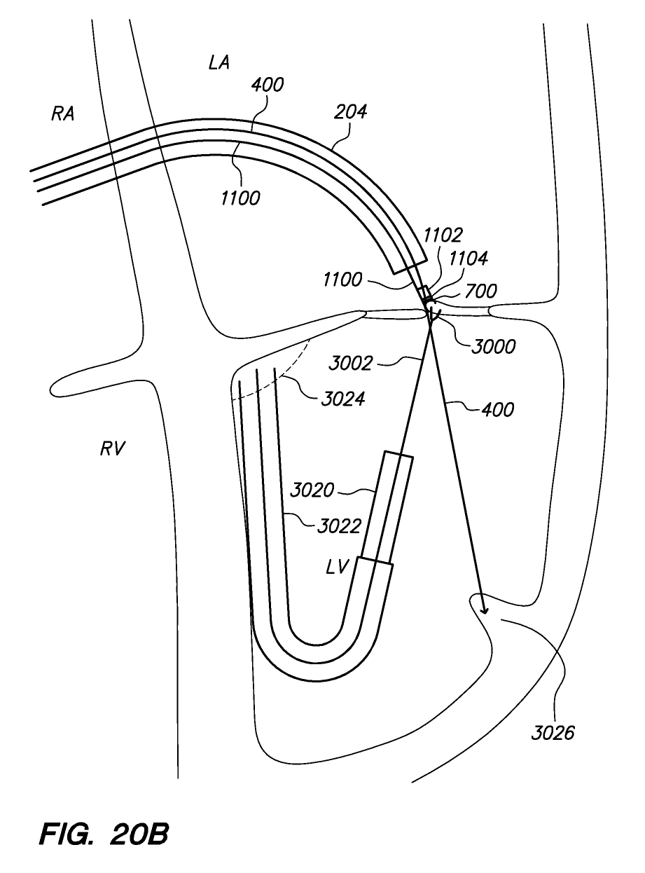

Referring to FIGS. 20A-20B, a combined antegrade and retrograde approach is depicted, wherein an antegrade deployment similar to that depicted in FIGS. 1-16C is shown, with the addition of leaflet distraction provided by a retrograde instrument having a grasping end effector. Referring to FIG. 20A, a papillary anchor (402) has been deployed with a trans-mitral approach and the transseptal delivery catheter (204) has been withdrawn back into the left atrium. A leaflet anchor (700) is positioned through the transseptal delivery catheter (204) utilizing an introducer (702). In one embodiment, as illustrated in FIG. 20B, the grasping tool (3000), controlled by a grasper proximal structure (3002), such as an introducer catheter or push or pull wire or probe, in addition to the retrograde delivery catheter complex (3020, 3022), is utilized to pull the targeted leaflet into a desired position while a fastening assembly (1100, 1102, 1104) is utilized from the antegrade approach to position the cord (400) for desirable valve mechanics. In other words, the retrograde instrumentation is utilized as a distraction apparatus to assist in the deployment. In another embodiment (not shown) wherein the leaflet anchor is not initially coupled to, or advanced over, the cord (400), the retrograde instrument assembly may comprise a clip applier rather than a simple grasper, and may be utilized to deploy a clip over the cord (400) and leaflet margin, as the cord (400) and leaflet margin intersect when the cord (400) is tensioned proximally toward the transseptal crossing.

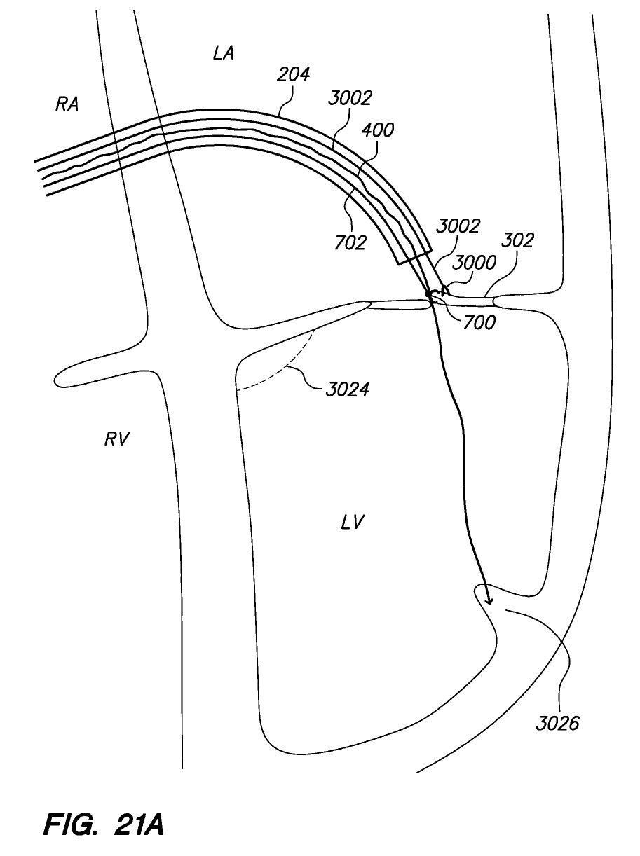

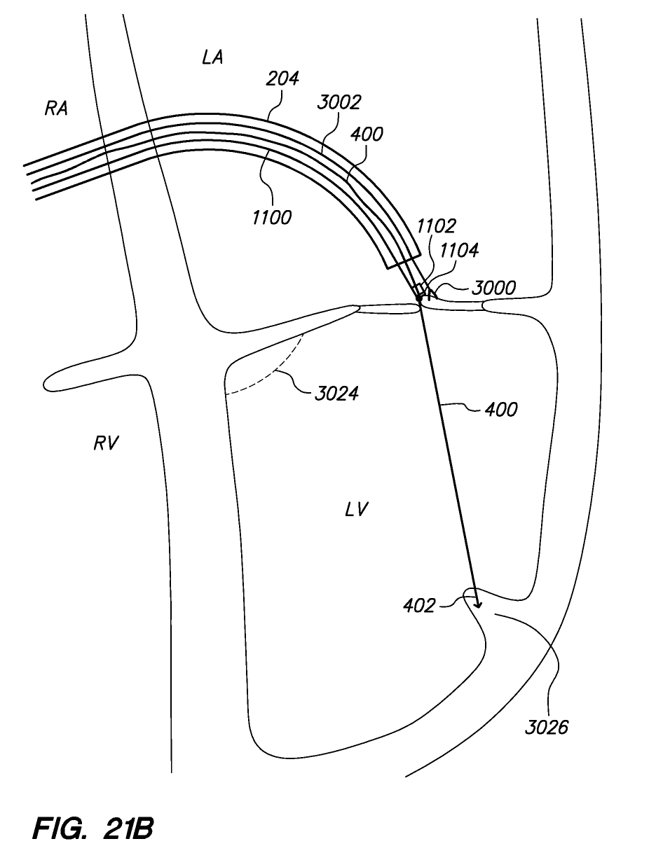

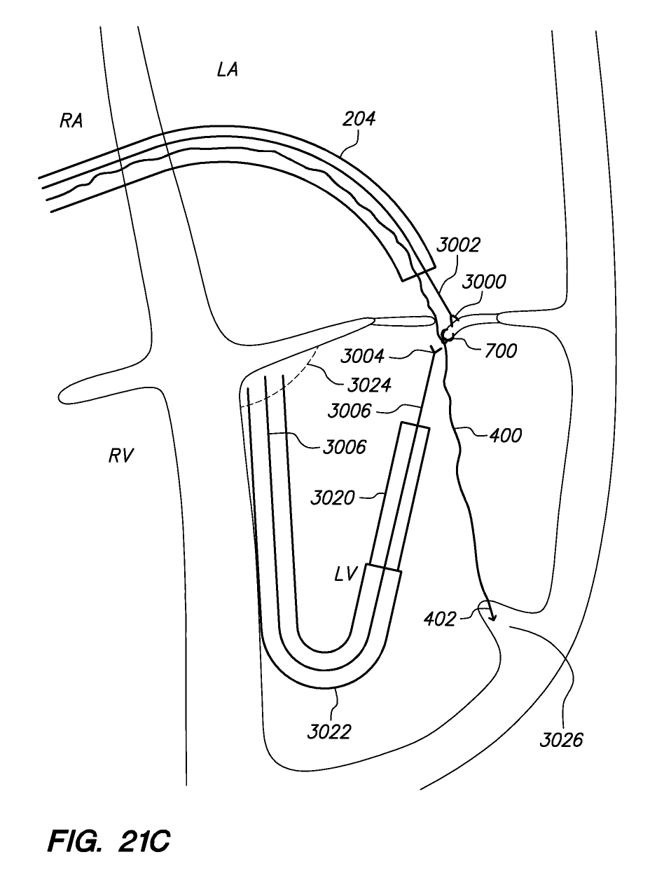

Referring to FIGS. 21A-21C, another combined antegrade/retrograde deployment embodiment is depicted. Referring to FIG. 21A, a papillary anchor (402) has been deployed with a trans-mitral approach from an antegrade transseptal catheter (204), and such catheter (204) has been withdrawn back to the left atrium. The papillary anchor (402) deployment introducer (404—not shown in this figure) has been withdrawn, and a grasping assembly comprising a grasper proximal structure (3002) and a leaflet grasper (3000) is being utilized to distract, or push, the targeted prolapsing leaflet (302) into a desirable position before permanent fastening of a leaflet anchor (700), which is shown attached to its introducer (702) in this illustration. Referring to FIG. 21B, a fastener complex (1100, 1102, 1104) is utilized to fasten the leaflet anchor (700) in place as the grasper (3000) is utilized to assist in the positioning of the leaflet and the cord (400) is pulled into a tension from an extracorporeal proximal location. Referring to FIG. 21C, in this embodiment, subsequent to deployment of the fastener (1104), the antegrade grasper (3000), grasper proximal structure (3002), and transseptal catheter (204) may be utilized to position the leaflet so that a retrograde assembly (3022, 3020, 3004, 3006) comprising a cutter or clipper (3004) and clipper proximal structure (3006, which may be similar to the aforementioned grasper proximal structure 3002) may be utilized to precisely address and sever the proximal cord remnant, as depicted in FIG. 21C.

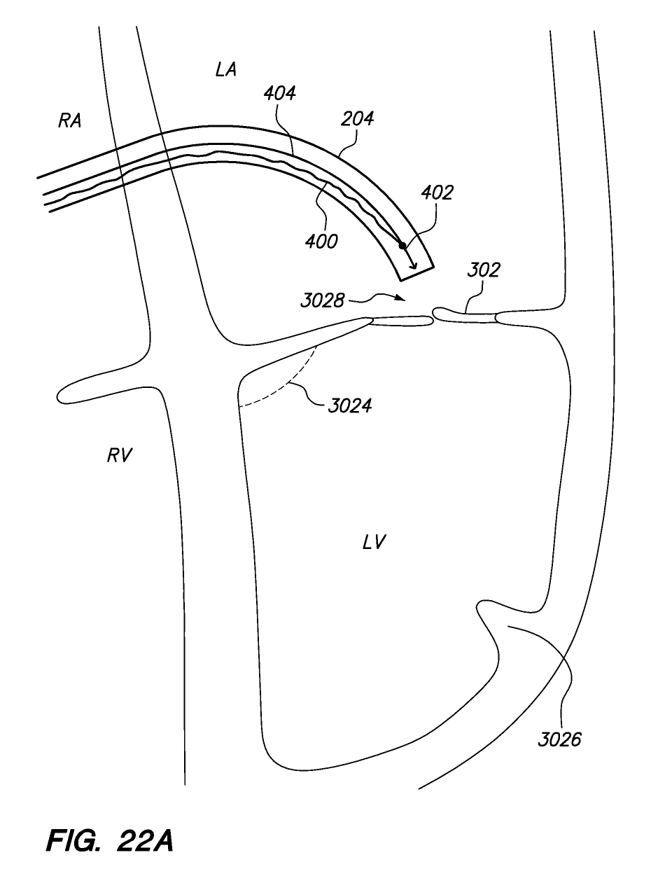

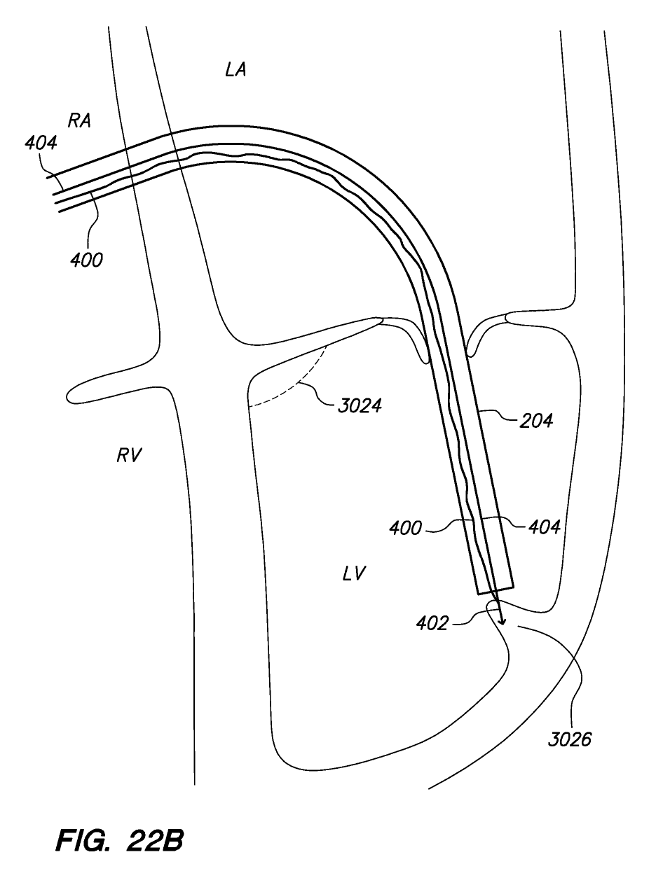

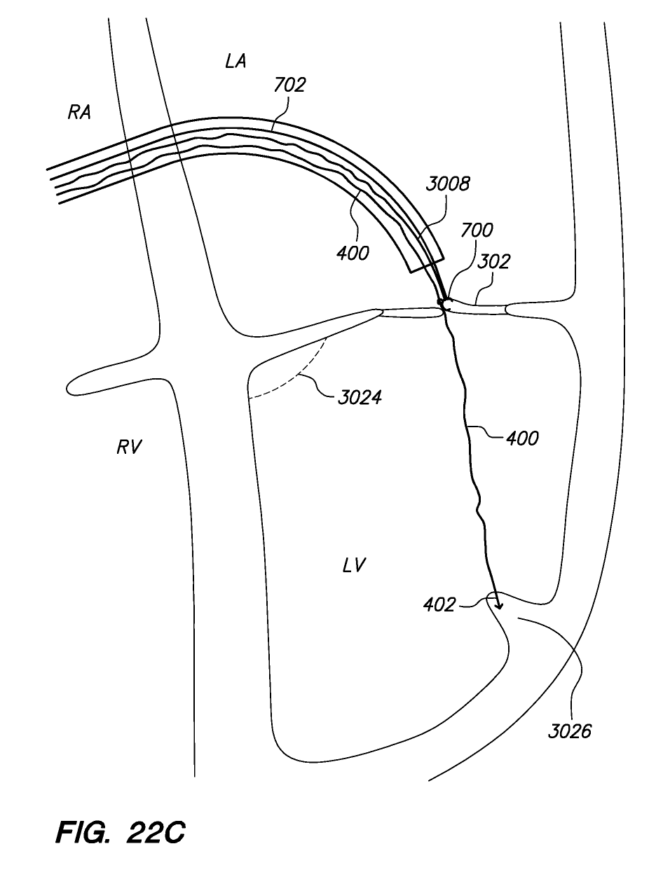

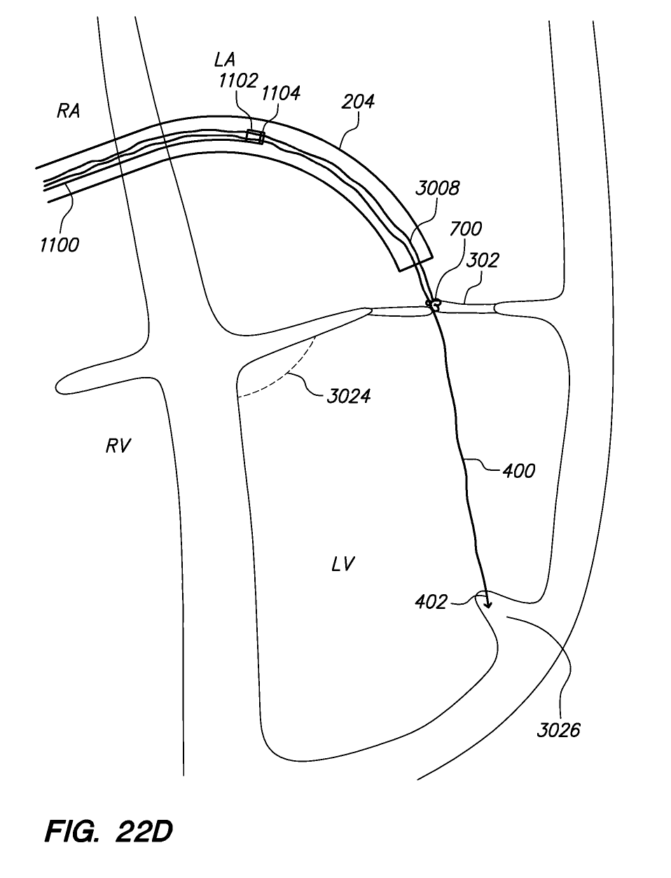

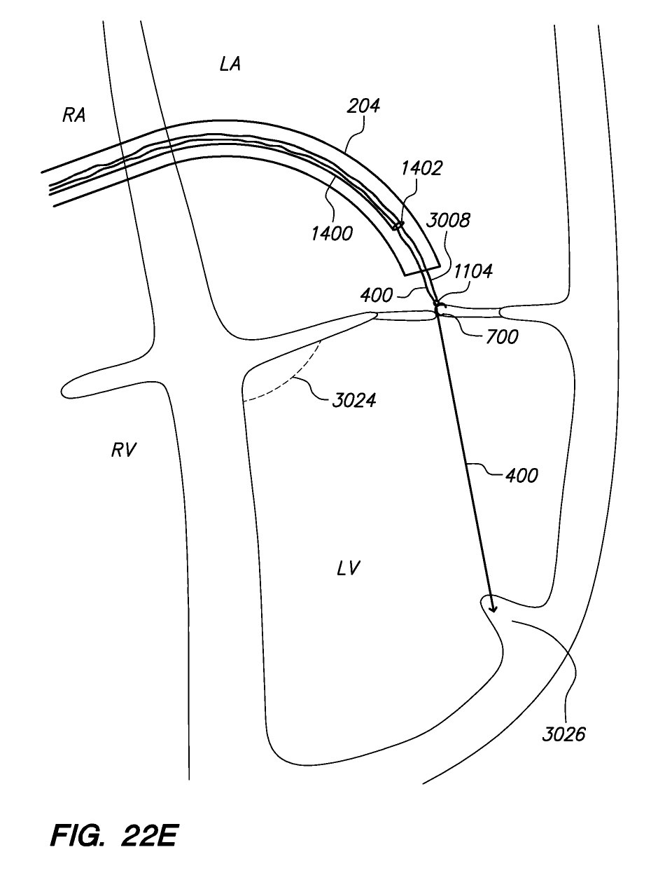

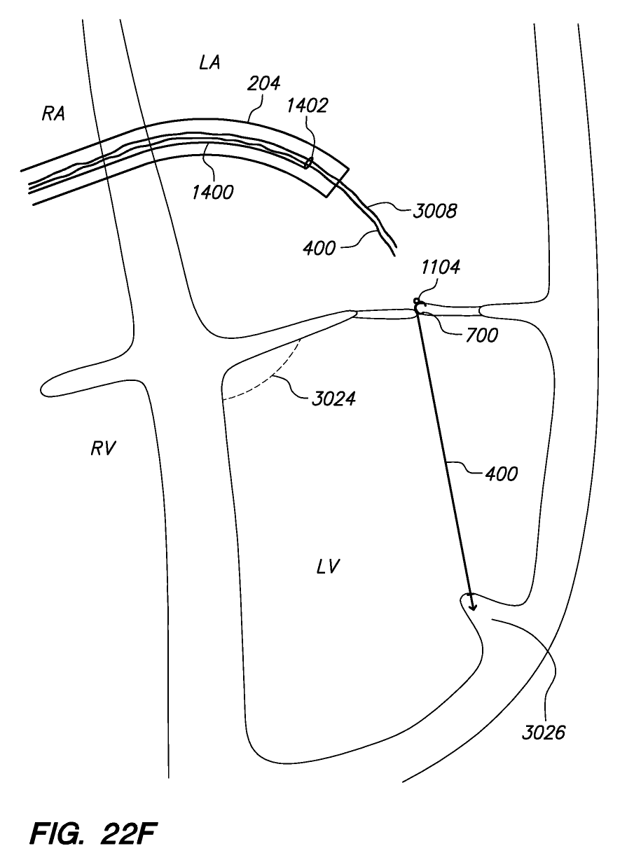

Referring to FIGS. 22A-22F, a double-cord embodiment is depicted wherein two anchors are deployed with independent cords running proximally, subsequent to which a fastener is utilized to join the cords and create a chordae prosthesis configuration. Referring to FIG. 22A, a papillary anchor (402) is being advanced toward the mitral valve (3028) from an antegrade approach. FIG. 22B depicts deployment of the papillary anchor (402) with an introducer (404) and the transseptal catheter (204). Referring to FIG. 22C, with the transseptal catheter (204) withdrawn back across the mitral valve into the left atrium, a leaflet anchor (700) is advanced utilizing an introducer (702) and having a separate cord (3008) running proximally. Referring to FIG. 22D, the leaflet anchor (700) has been deployed and the leaflet anchor introducer (702 in FIG. 22C) withdrawn proximally. A fastener assembly (1100, 1102, 1104) is advanced over both cords (400, 3008) and deployed to fasten both relative to the leaflet margin. Subsequently, a cutting assembly (1400, 1402) is utilized to trim the proximal cord remnants and leave a functional prosthetic assembly in place, as depicted in FIG. 22F.

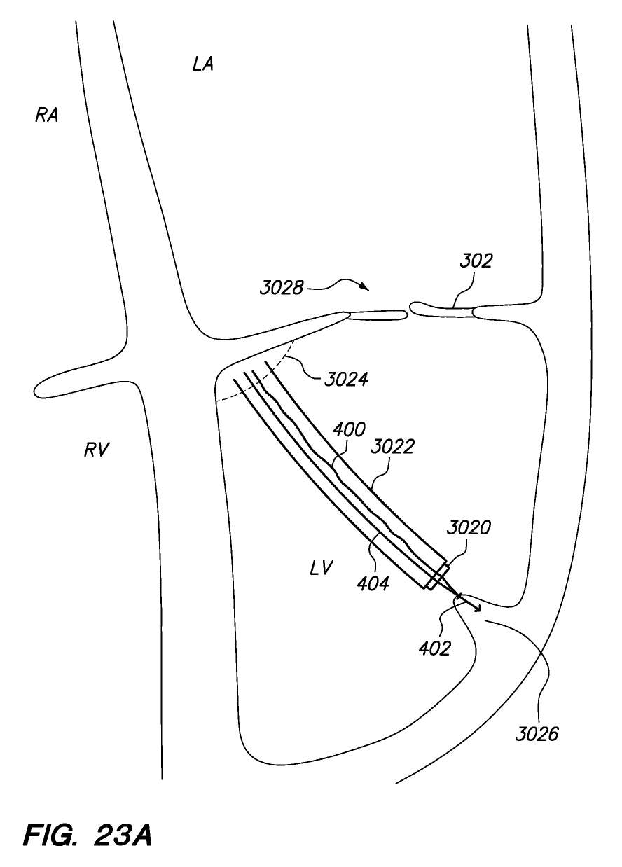

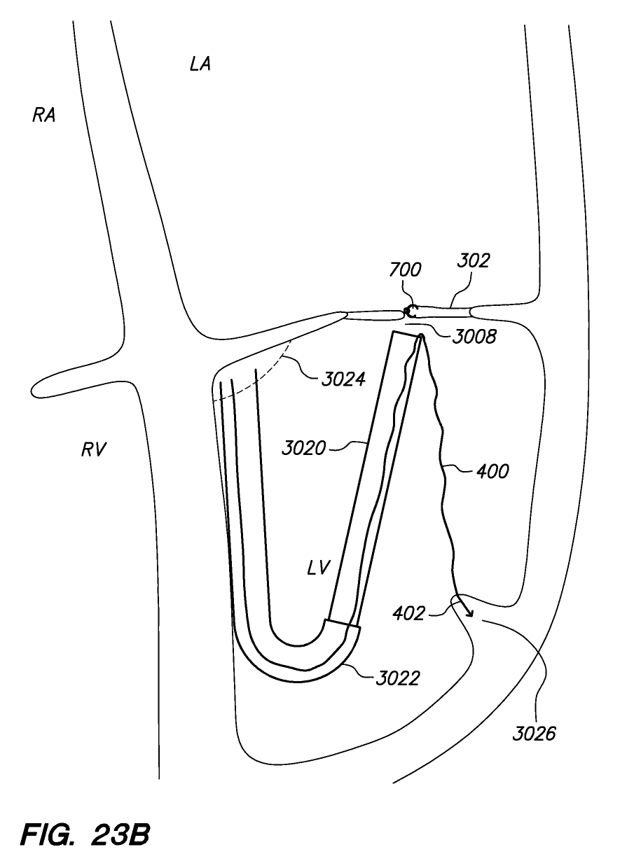

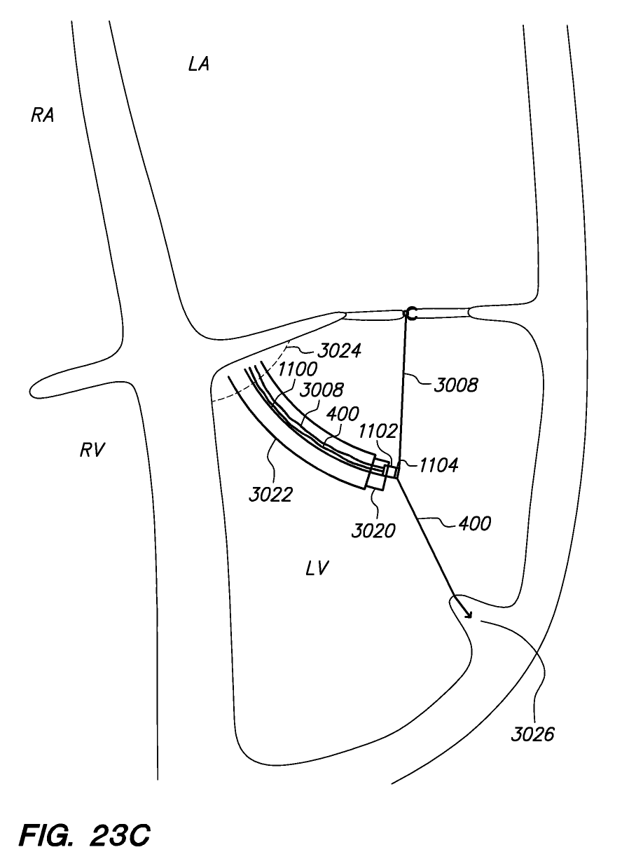

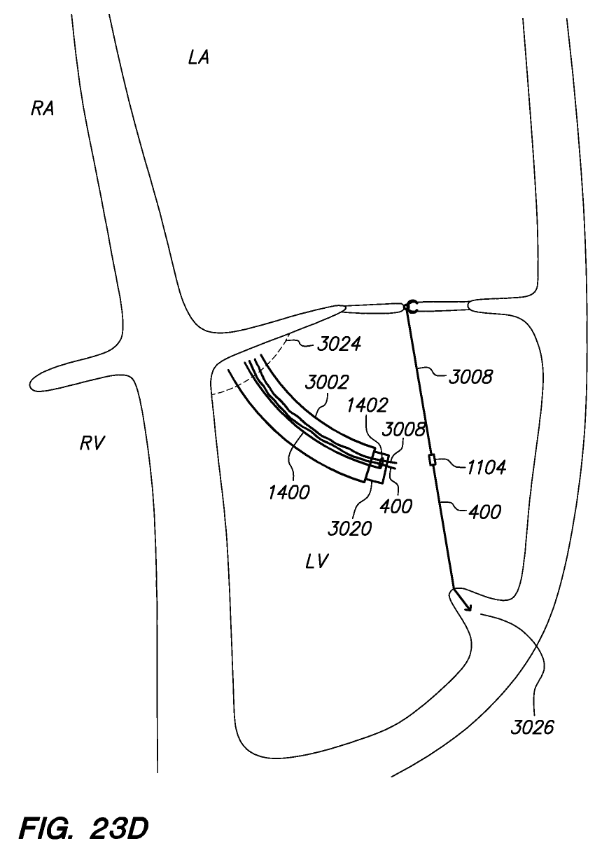

Referring to FIGS. 23A-23D, an alternative double-cord embodiment is depicted wherein two anchors are deployed with a retrograde approach, followed by a single fastener to join the two cords and create a functional prosthesis assembly to optimize mitral valve function. Referring to FIG. 23A, a retrograde catheter complex (3022, 3020) is depicted deploying a papillary anchor (402). The papillary anchor introducer (404) may be withdrawn, and the catheter complex (3020, 3022) distal portion navigated toward the targeted mitral leaflet margin, where a separately-corded leaflet anchor (700) may be deployed, as depicted in FIG. 23B. Referring to FIG. 23C, a fastener assembly (1100, 1102, 1104) and navigation of the catheter complex (3020, 3022) distal tip may be utilized to facilitate tensioning of the two cords (400, 3008) and deployment of the fastener (1104) to retain their position relative to each other and the pertinent tissue structures. Referring to FIG. 23D, a cutting assembly (1400, 1402) may be advanced over the proximal aspects of the cords to detract such proximal remnants and leave a functional prosthesis assembly (400, 3008, 1104, 700, 402) in place, as depicted in FIG. 23D.

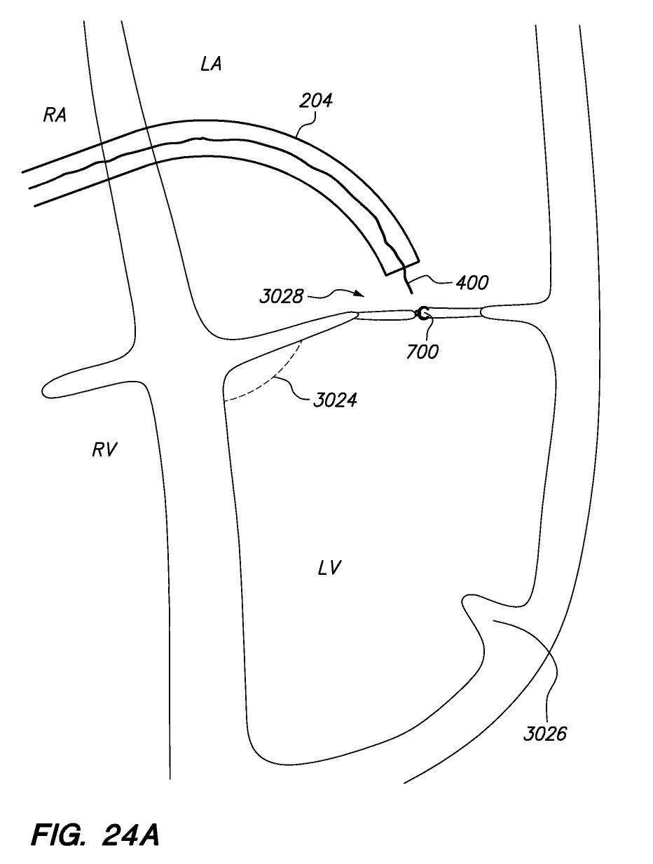

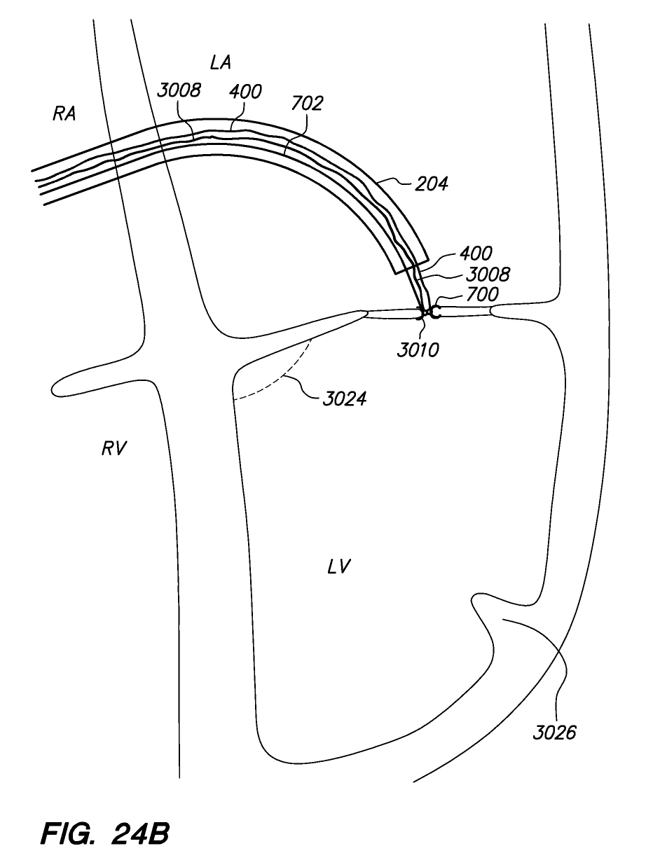

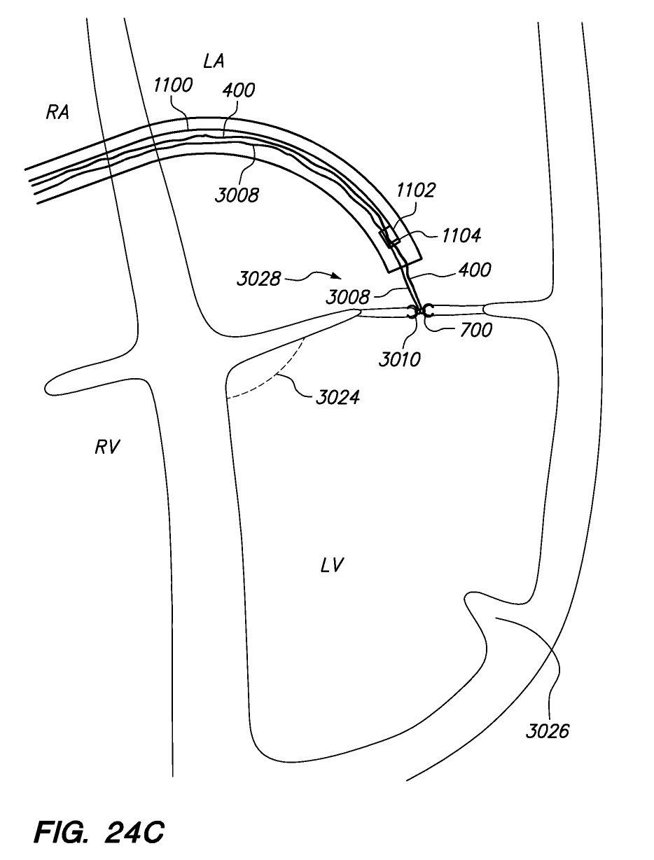

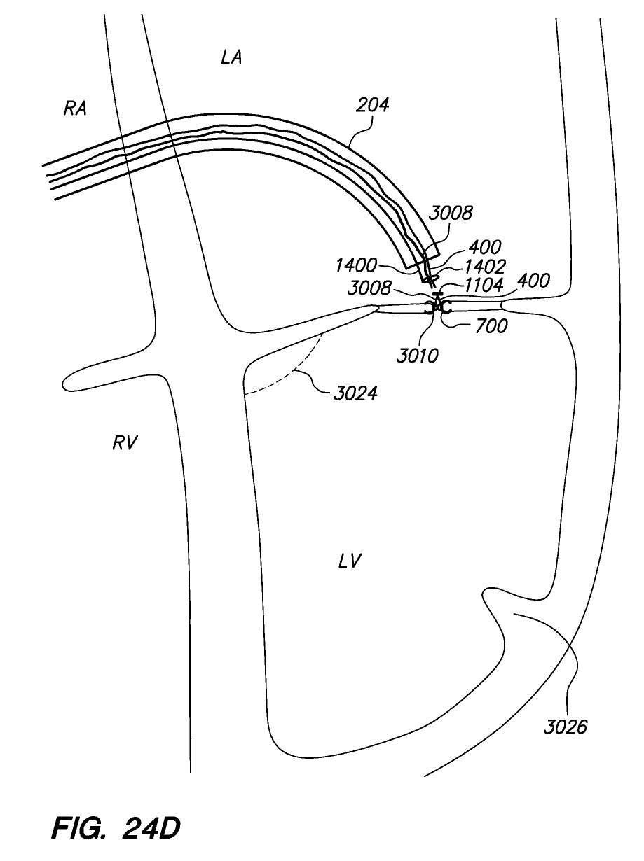

Referring to FIGS. 24A-24D, an Alfieri intervention is depicted using a pair of leaflet anchors and a double-cord deployment. Referring to FIG. 24A, an antegrade approach is utilized to deploy a first leaflet anchor (700) coupled to a first cord (400). Referring to FIG. 24B, a second leaflet anchor (3010) is deployed using an introducer (702—may be the same introducer that was previously used to deploy the first leaflet anchor 700—or may be a completely separate instrument) and having a second cord (3008). Referring to FIG. 24C, a fastener assembly (1100, 1102, 1104) is advanced over the cords (400, 3008). Referring to FIG. 24D, with the fastener (1104) deployed, a cutting assembly (1400, 1402) may be utilized to cut free the proximal remnants of the cords (400, 3008), leaving an Alfieri type prosthesis deployment configured to prevent or limit valve prolapse.

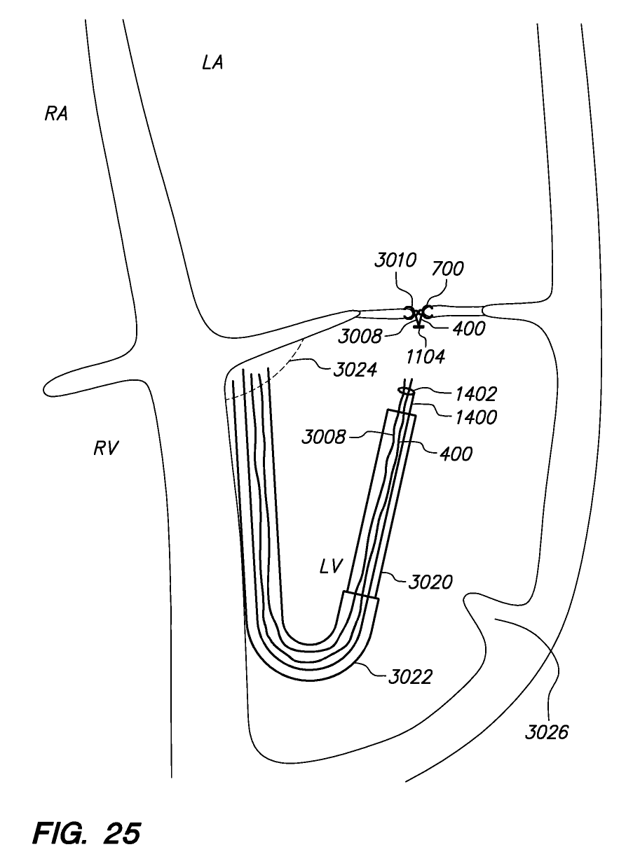

Referring to FIG. 25, a retrograde approach is depicted for forming an Alfieri type prosthesis deployment. A retrograde catheter assembly (3020, 3022) has been utilized to deliver a pair of leaflet anchors (700, 3010), each of which has its own cord (400, 3008). A fastening assembly (only the deployed fastener, 1104, shown) has been utilized to join the cords (400, 3008), and, as depicted in FIG. 25, a cutting assembly (1400, 1402) is being utilized to sever the remnant proximal aspects of these cords, leaving behind an Alfieri type deployment assembly configured to prevent or limit valve prolapse.

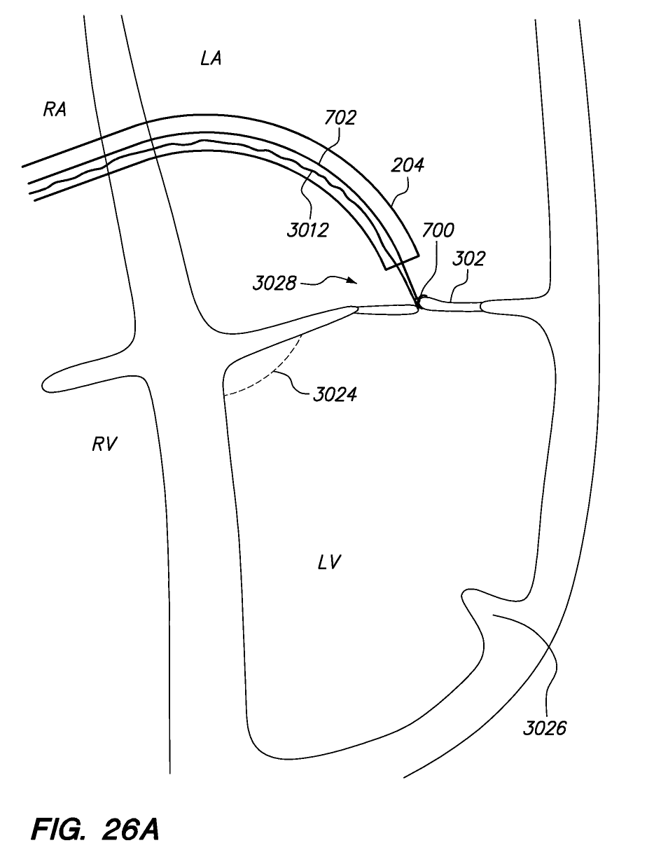

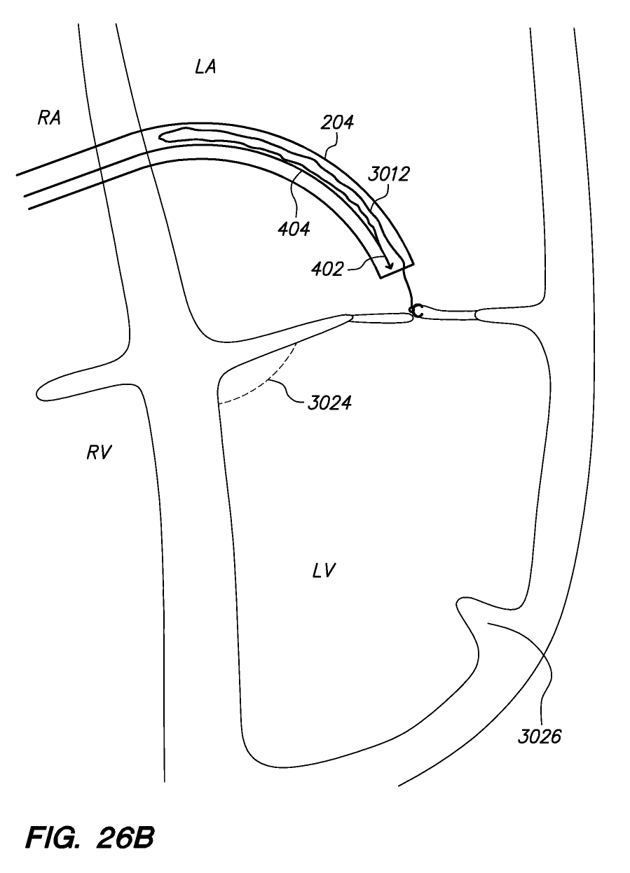

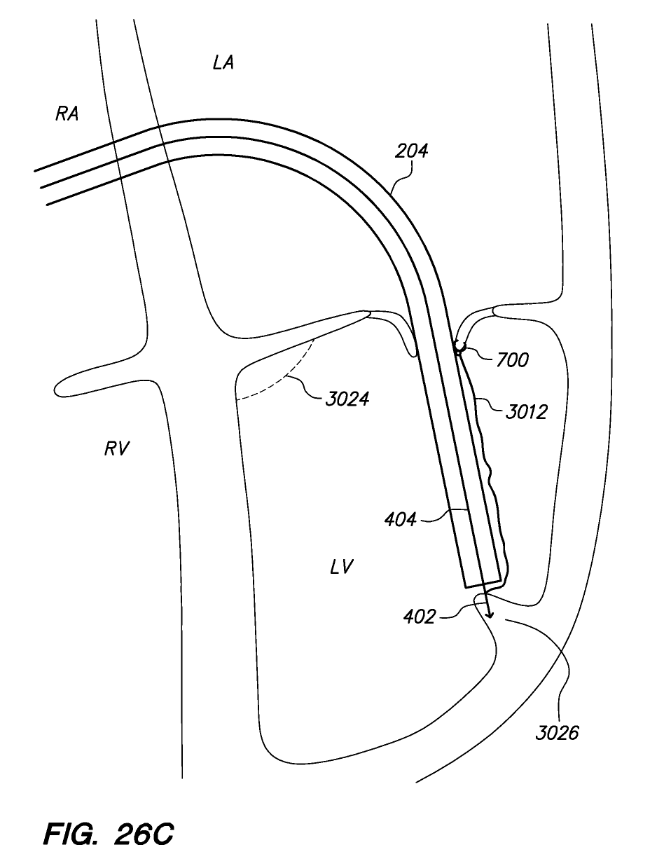

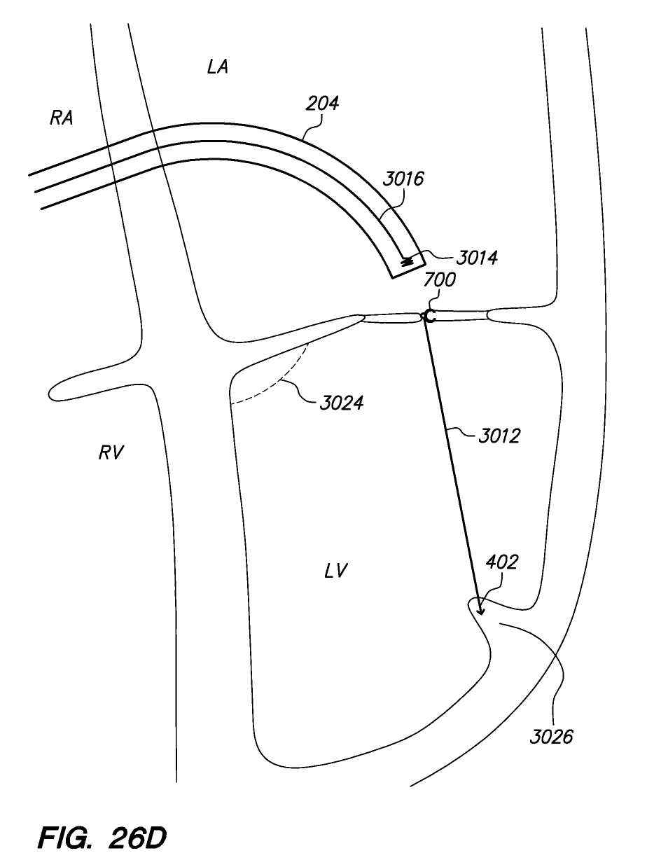

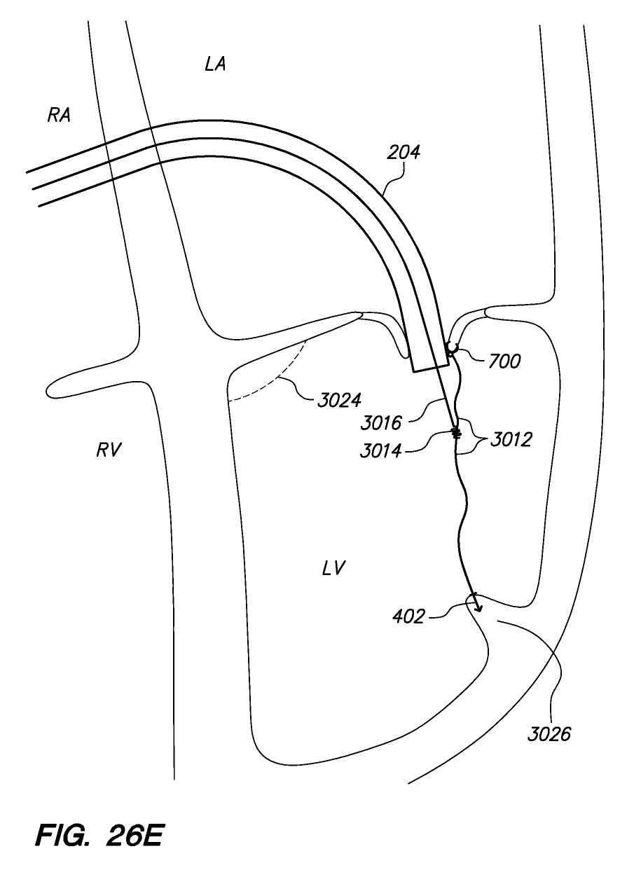

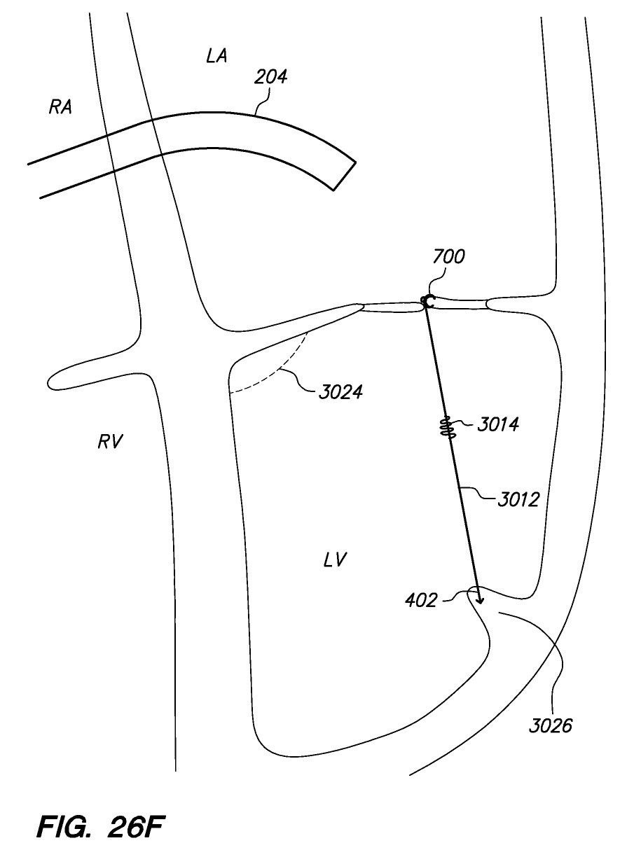

Referring to FIGS. 26A-26F, an embodiment utilizing a pre-measured length of cord (400) is depicted. Referring to FIG. 26A, an antegrade approach is utilized to deploy a leaflet anchor (700) to the margin of a prolapsing mitral leaflet (302) using an introducer (702) and transseptal catheter (204). Referring to FIG. 26B, after the leaflet anchor (700) has been deployed and the introducer withdrawn, a papillary anchor (402) is advanced with its introducer (404). The length of cord (3012) between the two anchors has been premeasured and predetermined utilizing information from anatomical imaging studies and an understanding of the valve and heart mechanics. Referring to FIG. 26C, the transseptal catheter (204) is advanced across the mitral valve to deploy the papillary anchor (402) with the associated introducer (404). Referring to FIG. 26D, after the papillary anchor introducer (404) has been released from the papillary anchor (402) and the transseptal catheter (204) has been withdrawn back into the left atrium, the premeasured cord and anchors assembly (700, 3012, 402) is deployed. In the event that additional tensioning is desired (for example, in one embodiment, the premeasured length is slightly increased to allow for fine tuning with additional incremental tensioning) a tensioning clip assembly, comprising a deployable tensioning clip (3014) configured to be fastened to the premeasured cord (3012) and to slightly decrease the length of such premeasured cord (3012), and also comprising a proximal delivery structure (3016, which may be similar to the aforementioned grasper proximal structure 3002, and deployable from the deployable tensioning clip (3014) in a manner analogous to the deployment of the aforementioned papillary and leaflet anchors from their introducers), is depicted and may be delivered utilizing movements of the transseptal catheter (204) and the proximal deliver structure (3016). FIGS. 26E and 26D depict deployment of a tensioning clip (3014) to fine tune the valve mechanics with a premeasured cord (3012) deployment. Additional increments of tensioning may be applied by deploying additional deployable tensioning clips (3014). Indeed, such an incremental tensioning adjustment schema may be utilized after any of the deployments described herein to fine tune tensioning.

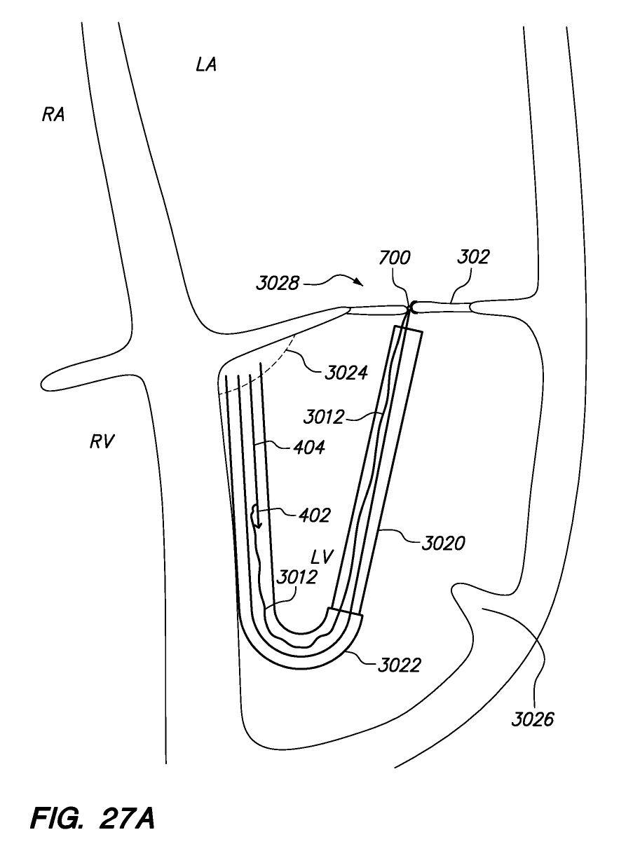

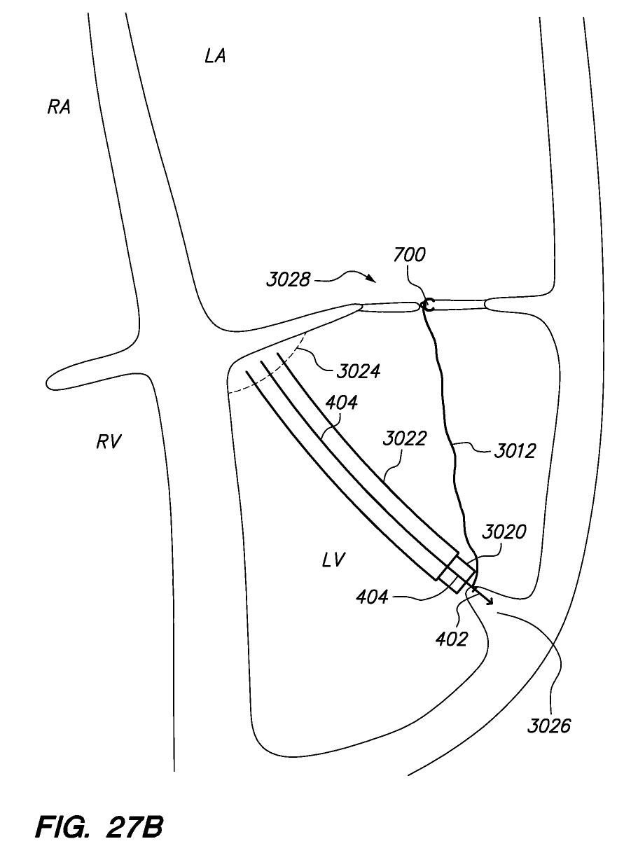

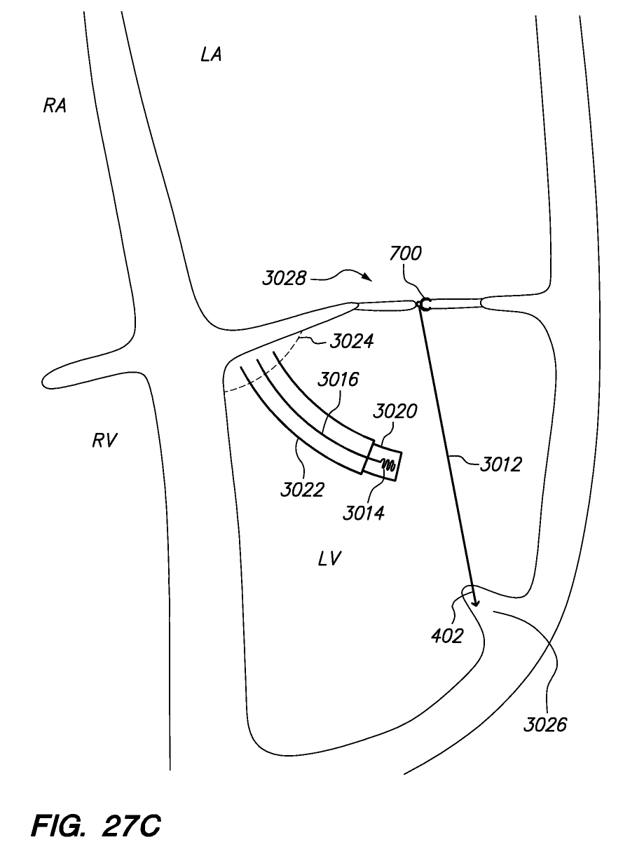

Referring to FIGS. 27A-27C, a retrograde approach utilizing a premeasured cord (3012) is depicted. Referring to FIG. 27A, a leaflet anchor (700) is deployed, followed by a papillary anchor (402), as depicted in FIG. 27B. Referring to FIG. 27C, after deployment of the papillary anchor (402) from its introducer (404 in FIG. 27B), the prosthesis assembly (700, 3012, 402) is deployed and may be fine-tuned utilizing a tensioning clip assembly (3014, 3016) and navigation of the retrograde catheter assembly (3020, 3022).

It should be noted that while the descriptions of the methods herein have focused on repair of mitral valve prolapse, the methods need not be so limited in application. For example, the methods may be used to correct tricuspid valve prolapse, or to provide a tensioned cord between any two areas of tissue requiring such. In addition, while the methods described here focused on a percutaneous trans-septal access, any method of accessing the prolapsed valve is suitable, as noted above.

### Kits

Kits for treating a mitral valve prolapse are also provided. In general, the kits comprise a flexible cord, having a proximal end and a distal end, a first anchor attached to the cord at its distal end and configured to secure the cord to cardiac tissue located below the prolapsed mitral valve leaflet, a second anchor for slidable attachment to the cord, wherein the second anchor is configured to secure into a the prolapsed mitral valve leaflet, and at least one catheter for delivery of the flexible cord to the proximity of the prolapsed mitral valve leaflet. Additional catheters may be included in the kits as well.

Any number of appropriate catheters may be used with devices and methods described herein, and may be included as part of a kit. For example, individual catheters may be used to deliver and/or implant the first anchor and cord, to delivery and/or implant the second anchor, to reposition the valve leaflet, or to deliver and/or secure a fastener for the cord. Additional catheters may also be included. In some variations, catheters may be combined. For example, a single catheter may be used to deliver and/or implant the second anchor and to reposition the leaflet. Thus, any of the catheters may be combined.

In addition, as evidenced by the description of the methods above, it may be advantageous to provide a kit with additional tools useful in carrying out the described methods. For example, the kits may further comprise a cutting wire for cutting the cord, and/or a fastener. The kit may also include instructions on how to use the contents of the kit. Instructions may include reference materials (including indications for use, etc.) and be in any appropriate format, including written, pictographic, visual, electronic, etc., and be in any language, or multiple languages.

As with the devices described above, at least one of the first or second anchors may be made from a shape memory material, such as a nickel titanium alloy. The first anchor may have a tissue piercing tip, which may further comprise two legs that may or may not be configured to expand into cardiac tissue. Similarly, the cord may be made from a material selected from the group consisting of non-polymeric fabrics, polymers, and mixtures thereof. In some variations, the cord is made from a non-polymeric fabric and polymer mixture, such as a PTFE fabric.

Although specific embodiments of the present invention have been illustrated in the accompanying drawings and described in the foregoing detailed description, it will be understood that the invention is not limited to the particular embodiments described herein, but is capable of numerous rearrangements, modifications, and substitutions without departing from the scope of the invention.

1. A system for deploying a tensile member between two tissue structures, comprising:
a. a first anchor insertion instrument defining a working lumen therethrough; and
b. a second anchor insertion instrument movably coupled through said working lumen;
wherein the first anchor instrument defines a jaw space and is configured to deploy a staple prosthesis into a portion of a first tissue structure which may be captured within said jaw space;
and wherein the second anchor instrument is configured to deploy an implantable anchor into a second tissue structure which is coupled to the staple prosthesis by a tensile member.

2. The system of claim 1, wherein the jaw space of the first anchor insertion instrument is defined distally by an anvil structure.

3. The system of claim 1, wherein the jaw space of the first anchor insertion instrument is defined proximally by a structure selected from the group consisting of a proximal stapler housing, an advanceable stapling member, and an advanceable jaw member.

4. The system of claim 3, wherein portion of the first tissue structure may be controllably compressed by the advanceable jaw member before a staple is advanced through such first tissue structure portion by the advanceable stapling member, thereby driving the staple through the portion of the first tissue structure.

5. The system of claim 1, wherein the implantable anchor comprises an anchor dart having a distal body portion and a plurality of legs defining a proximal portion.

6. The system of claim 5, wherein a lumen is defined through said body portion.

7. The system of claim 6, wherein a working space is defined in between the plurality of legs.

8. The system of claim 7, wherein the second anchor insertion instrument comprises an elongate probe having a distal end removably inserted through the working space and body portion lumen of the anchor dart.

9. The system of claim 5, wherein the second anchor insertion instrument comprises an elongate probe defining a working lumen through which the anchor dart may be advanced into the second tissue structure.

10. The system of claim 9, wherein the distal portion of the elongate probe comprises a sharp geometry configured for atraumatic advancement through the second tissue structure before deployment of the anchor dart.

11. A method for deploying a tensile member between two tissue structures, comprising:
a. navigating a delivery instrument assembly defining a working lumen to a position adjacent a first targeted tissue structure;
b. deploying an implantable anchor dart prosthesis into the first targeted tissue structure, the anchor dart prosthesis being coupled to a tensile member which remains coupled to the delivery instrument assembly;
c. navigating the delivery instrument assembly to a position adjacent a second targeted tissue structure;
d. deploying a second anchor into the second targeted tissue structure which remains coupled to the anchor dart prosthesis by the tensile member;
e. adjusting the length of the tensile member between the anchor dart and second anchor; and
f. removing the delivery instrument assembly.

12. The method of claim 11, wherein the first targeted tissue structure is a portion of a papillary muscle.

13. The method of claim 11, wherein the second targeted tissue structure is a portion of a mitral valve leaflet.

14. The method of claim 11, wherein navigating a delivery instrument comprises operating a remotely steerable catheter.

15. The method of claim 11, wherein deploying an implantable anchor dart prosthesis comprises inserting a deployment probe having a sharp distal tip and a working lumen in which the implantable anchor dart prosthesis is disposed.

16. The method of claim 15, wherein deploying an implantable anchor dart prosthesis further comprises advancing the anchor dart prosthesis distally relative to the deployment probe.

17. The method of claim 11, wherein deploying an implantable anchor dart prosthesis comprises inserting a deployment probe having a sharp distal tip positioned through a proximal workspace and body lumen defined by the anchor dart prosthesis, such that the sharp distal tip extends beyond a distal tip of the anchor dart prosthesis.

18. The method of claim 17, wherein deploying an implantable anchor dart prosthesis further comprises inserting the deployment probe sharp distal tip into the first targeted tissue structure with a relatively high impulse load/timing profile.

19. The method of claim 18, wherein deploying an implantable anchor dart prosthesis further comprises withdrawing the deployment probe proximally relative to the implantable anchor dart.

20. The method of claim 1, wherein deploying a second anchor comprises deploying a staple.
